(19) United States
(12) Patent Application Publication
(10) Pub. No.: US 2006/0223868 A1
(43) Pub. Date: Oct. 5, 2006

(54) Heterocyclic amides exhibiting and inhibitory activity at the vanilloid receptor 1(vr1)

(75) Inventors: Yevgeni Besidski, Sodertalje (SE); William Brown, St. Laurent (CA); Shawn Johnstone, St. Laurent (CA); Denis Labrecque, St. Laurent (CA); Alexander Munro, St. Laurent (CA); Didier Rotticci, Sodertalje (SE); Christopher Walpole, St. Laurent (CA); Ronald Zemribo, Riga (LV); Andreas Petersson, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 10/554,036

(22) PCT Filed: Apr. 26, 2004
(86) PCT No.: PCT/SE04/00635

§ 371 Date: Oct. 20, 2005

(30) Foreign Application Priority Data
Apr. 28, 2003 (SE) 0301246-5
May 5, 2003 (SE) 0301305-9
Jan. 12, 2004 (SE) 0400044-4

(51) Int. Cl. A61K 31/428; A61K 31/423; A61K 31/4184

(52) U.S. Cl. 514/367; 514/375; 514/394; 548/152; 548/304.4; 548/217

(57) ABSTRACT

Compounds of formula I, 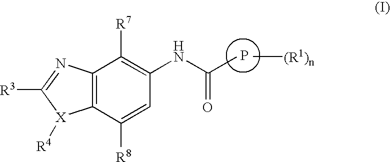
wherein R¹, n, R³, R⁴, R⁷, R⁸ and P are as defined in the specification, salts, solvates or solvated salts thereof, processes for their preparation, new intermediates used in the preparation thereof, pharmaceutical compositions containing said compounds and the use of said compounds in therapy.

### FIELD OF THE INVENTION

The present invention relates to new compounds, to pharmaceutical compositions containing said compounds and to the use of said compounds in therapy. The present invention further relates to processes for the preparation of said compounds and to new intermediates used in the preparation thereof.

### BACKGROUND OF THE INVENTION

Pain sensation in mammals is due to the activation of the peripheral terminals of a specialized population of sensory neurons known as nociceptors. Capsaicin, the active ingredient in hot peppers, produces sustained activation of nociceptors and also produces a dose-dependent pain sensation in humans. Cloning of the vanilloid receptor 1 (VR1 or TRPV1) demonstrated that VR1 is the molecular target for capsaicin and its analogues. (Caterina, M. J., Schumacher, M. A., et. al. Nature (1997) v. 389 p 816-824). Functional studies using VR1 indicate that it is also activated by noxious heat, tissue acidification) and other inflammatory mediators (Tominaga, M., Caterina, M. J. et. al. Neuron (1998) v. 21, p. 531-543). Expression of VR1 is also regulated after peripheral nerve damage of the type that leads to neuropathic pain. These properties of VR1 make it a highly relevant target for pain and for diseases involving inflammation. While agonists of the VR1 receptor can act as analgesics through nociceptor destruction, the use of agonists, such as capsaicin and its analogues, is limited due to their pungency, neurotoxicity and induction of hypothermia. Instead, agents that block the activity of VR1 should prove more useful. Antagonists would maintain the analgesic properties, but avoid pungency and neurotoxicity side effects. Compounds with VR1 inhibitor activity are believed to be of potential use for the treatment and/or prophylaxis of disorders such as pain, especially that of inflammatory or traumatic origin such as arthritis, ischaemia, cancer, fibromyalgia, low back pain and post-operative pain (Walker et al J Pharmacol Exp Ther. January 2003;304(1):56-62). In addition to this visceral pains such as chronic pelvic pain, cystitis, irritable bowel syndrome (IBS), pancreatitis and the like, as well as neuropathic pain such as sciatia, diabetic neuropathy, HIV neuropathy, multiple sclerosis, and the like (Walker et al ibid, Rashid et al J Pharmacol Exp Ther. March 2003;304(3):940-8), are potential pain states that could be treated with VR1 inhibiton These compounds are also believed to be potentially useful for inflammatory disorders like asthma, cough, inflammatory bowel disease (IBD) (Hwang and Oh Curr Opin Pharmacol June 2002;2(3):235-42). Compounds with VR1 blocker activity are also useful for itch and skin diseases like psoriasis and for gastro-esophageal reflux disease (GERD), emesis, cancer, urinary incontinence and hyperactive bladder (Yiangou et al BJU Int June 2001;87(9):774-9, Szallasi Am J Clin Pathol (2002) 118: 110-21). VR1 inhibitors are also of potential use for the treatment and/or prophylaxis of the effects of exposure to VR1 activators like capsaicin or tear gas, acids or heat (Szallasi ibid).

A further portential use relates to the treatment of tolerance to VR1 activators. VR1 inhibitors may also be useful in the treatment of interstitial cystitis and pain related to interstitial cystitis.

### DETAILED DESCRIPTION OF THE INVENTION

The object of the present invention is to provide compounds exhibiting an inhibitory activity at the vanilloid receptor 1 (VR1).

The present invention provides a compound of formula I

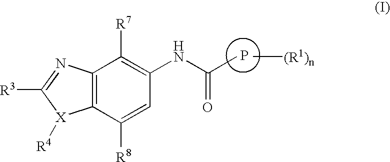

wherein:

ring P is C₆₋₁₀aryl, C₃₋₇cycloalkyl, C₅₋₆heteroaryl, which ring P may be fused with phenyl, C₅₋₆heteroaryl, C₃₋₇cycloalkyl or C₃₋₇heterocycloalkyl;

R¹ is NO₂, NH₂, halo, N(C₁₋₆alkyl)₂, C₁₋₆alkyl, C₂₋₆alkenyl, C₂₋₆alkynyl, C₁₋₆haloalkyl, C₁₋₆haloalkylO, phenylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆alkylOC₀₋₆alkyl, C₁₋₆alkylSC₀₋₆alkyl or C₁₋₆alkylNC₀₋₆alkyl;

n is 1, 2, 3, 4 or 5;

X is O or S, when

R³ is H, C₁₋₆alkyl, C₁₋₆haloalkyl, R⁵OC₁₋₆alkyl, R⁵OCO, R⁵CO, NR⁵R⁶CO, NR⁵R⁶C₀₋₆alkyl, C₂₋₆alkenylOC₀₋₆alkyl or hydroxyC₁₋₆alkyl; and

R⁴ is nil; or

X is N, when

R³ is H, C₁₋₆alkyl, C₁₋₆iodoalkyl, C₁₋₆bromoalkyl, C₁₋₆chloroalkyl, C₁₋₆alkylOC₀₋₆alkyl, R⁵OC₁₋₆alkyl, R⁵CO, R⁵CO2, NR⁵R⁶CO, NR⁵R⁶C₀₋₆alkyl or C₂₋₆alkenylOC₀₋₆alkyl; and

R⁴ is H, C₁₋₄alkyl, hydroxyC₁₋₆alkyl or C₁₋₆alkylOC₁₋₆alkyl; or

X is N, when R³ is C₁₋₆fluoroalkyl or hydroxyC₁₋₂alkyl and R⁴ is H;

R⁵ and R⁶ are independently selected from H, C₁₋₆alkyl, C₆₋₁₀aryl, C₅₋₆heteroaryl, C₁₋₄alkylSO₂ and C₁₋₃ alkylCO;

R⁷ and R⁸ are independently selected from H, C₁₋₆alkyl, halo, cyano, C₁₋₆alkylOC₀₋₆alkyl, OH, NO₂ and COR⁹, N(R⁹)₂;

R⁹ is H or C₁₋₆alkyl;

and wherein any alkyl, alkylOalkyl, haloalkyl, haloalkylO, phenyl, heteroaryl, cycloalkyl or heterocycloalkyl group may be substituted with one or more A; and

A is OH, NO₂, C₁₋₆alkylCO, C₁₋₆alkylO(CO), N(R⁹)₂, R⁹S, R⁹SO₂, halo or C₁₋₆alkylOC₀₋₆alkyl,

or salts, solvates or solvated salts thereof.

One embodiment of the invention relates to the compound of formula I wherein ring P is C₆₋₁₀aryl, C₅₋₆heteroaryl, which ring P may be fused with C₃₋₇heterocycloalkyl;

R¹ is NO₂, NH₂, halo, N(C₁₋₆alkyl)₂, C₁₋₆alkyl, C₂₋₆alkenyl, C₁₋₆haloalkyl, C₁₋₆haloalkylO, phenylC₀₋₆alkyl, C₃₋₇heterocycloalkylC₀₋₆alkyl, C₁₋₆alkylOC₀₋₆alkyl or C₁₋₆alkylSC₀₋₆alkyl;

n is 1, 2 or 3;

X is O or S, when

R³ is C₁₋₆alkyl, NR⁵R⁶CO, NR⁵R⁶C₀₋₆alkyl, C₂₋₆alkenylOC₀₋₆alkyl or hydroxyC₁₋₆alkyl; and

R⁴ is nil; or

X is N, when

R³ is H or C₁₋₆alkyl; and

R⁴ is C₁₋₄alkyl or hydroxyC₁₋₆alkyl; or

X is N, when R³ is C₁₋₆fluoroalkyl and R⁴is H;

R⁵ and R⁶ are independently selected from H, C₆₋₁₀aryl, C₅₋₆heteroaryl, C₁₋₄alkylSO₂ and C₁₋₃ alkylCO;

R⁷ and R⁸ are independently selected from H, halo and cyano;

and wherein any alkyl, phenyl, heteroaryl group may be substituted with one or more A; and

A is OH, NO₂, halo or C₁₋₄alkylOC₀₋₆alkyl;

or salts, solvates or solvated salts thereof.

In one embodiment of the invention X is S and R³is C₁₋₆alkyl, NR⁵R⁶CO, NR⁵R⁶C₀₋₆alkyl, C₂₋₆alkenylOC₀₋₆alkyl or hydroxyC₁₋₆alkyl.

In another embodiment X is S and R³is methyl.

In a further embodiment X is S and R³is hydroxymethyl.

In one embodiment of the invention X is O and R³ is C₁₋₆alkyl or hydroxyC₁₋₆alkyl.

In another embodiment X is O and R³ is methyl.

In a further embodiment X is O and R³ is hydroxymethyl.

In one embodiment of the invention X is N and R³ is C₁₋₆alkyl and R⁴ is C₁₋₆alkyl or hydroxyC₁₋₆alkyl.

In another embodiment R³ is methyl and R⁴is methyl or 2-hydroxyethyl.

In a further embodiment X is N and R³ is trifluoromethyl and R⁴is H

R⁵ and R⁶ may optionally be substituted by A. In one embodiment R⁵ and R⁶ are selected independently from the group consisting of H, methylsulfonyl, acetyl and substituted or unsubstituted heteroaryl such as pyrazole or pyridine.

One embodiment of the invention relates to the compound of formula I wherein R³ is hydroxymethyl, allyloxymethyl, ethoxymethyl, methoxypyridinylaminomethyl, pyrazolylaminomethyl, aminomethyl, methylsulfonylaminomethyl, acetylaminomethyl, carboxamide, methyl, hydroxyethyl, nitrophenylaminomethyl, hydroxycarbonyl or methoxycarbonyl.

R⁴ may be selected from the group consisting of H, C₀₋₄alkyl or hydroxyC₁₋₆alkyl.

In one embodiment of the invention P is substituted with 0, 1, 2, 3 or 4 groups R¹, wherein the number of R¹ substituents on the P ring is designated by the term n. In another embodiment of the invention n is 1 or 2.

Another embodiment of the invention relates to the compound of formula I wherein ring P is phenyl.

In a further embodiment ring P is phenyl and R¹ is NO₂, NH₂, halo, N(C₁₋₆alkyl)₂, C₁₋₆alkyl, C₂₋₆alkenyl, C₂₋₆alkynyl, C₁₋₆haloalkyl, C₁₋₆haloalkylO, phenylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆alkylOC₀₋₆alkyl, C₁₋₆alkylSC₀₋₆alkyl or C₁₋₆alkylNC₀₋₆alkyl optionally substituted with one or more A.

In yet another embodiment ring P is pyrazolyl, pyridine, benzdioxolane, furan, thiophene or naphthalene and R¹ is NO₂, NH₂, halo, N(C₁₋₆alkyl)₂, C₁₋₆alkyl, C₂₋₆alkenyl, C₂₋₆alkynyl, C₁₋₆haloalkyl, C₁₋₆haloalkylO, phenylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆alkylOC₀₋₆alkyl, C₁₋₆alkylSC₀₋₆alkyl or C₁₋₆alkylNC₀₋₆alkyl optionally substituted with one or more A.

Ring P may be substituted by R¹ on a nitrogen or carbon atom in ring P. Further, one atom on ring P may be substituted by two substituents R¹.

Any alkyl, alkylOalkyl, haloalkyl, haloalkylO, phenyl, heteroaryl, cycloalkyl or heterocycloalkyl group present in the substituents of the compounds of formula I may be substituted with one or more A. One embodiment of the invention relates to compounds of formula I wherein A is selected from the group consisting of OH, NO₂, halo or C₁₋₆alkylOC₀₋₆alkyl.

Another embodiment of the invention relates to compounds selected from the group consisting of

3-Fluoro-N-(2-methyl-1,3-benzothiazol-5-yl)-4-trifluoromethyl-benzamide,

2-tert-Butyl-5-methyl-2H-pyrazole-3-carboxylic acid (2-methyl-1,3-benzothiazol-5-yl)-amide,

2-Fluoro-N-(2-methyl-1,3-benzothiazol-5-yl)-4-trifluoromethyl-benzamide,

2-Fluoro-N-(2-methyl-1,3-benzothiazol-5-yl)-3-trifluoromethyl-benzamide,

4-Fluoro-N-(2-methyl-1,3-benzothiazol-5-yl)-3-trifluoromethyl-benzamide,

3,4-Dimethyl-N-(2-methyl-benzothiazol-5-yl)-benzamide,

2,2-Difluoro-benzo[1,3]dioxole-5-carboxylic acid (2-methyl-1,3-benzothiazol-5-yl)-amide,

N-(2-Methyl-1,3-benzothiazol-5-yl)-6-trifluoromethyl-nicotinamide,

N-(2-Methyl-1,3-benzothiazol-5-yl)-4-propyl-benzamide,

3-Iodo-N-(2-methyl-1,3-benzothiazol-5-yl)-benzamide,

2,5-Dimethyl-furan-3-carboxylic acid (2-methyl-1,3-benzothiazol-5-yl)-amide,

5-tert-Butyl-2-methyl-furan-3-carboxylic acid (2-methyl-1,3-benzothiazol-5-yl)-amide,

4-Bromo-3-methyl-N-(2-methyl-1,3-benzothiazol-5-yl)-benzamide,

3,4-Difluoro-N-(2-methyl-1,3-benzothiazol-5-yl)-benzamide,

3-Chloro-2-fluoro-N-(2-methyl-1,3-benzothiazol-5-yl)-benzamide,

Pyridine-2-carboxylic acid (2-methyl-1,3-benzothiazol-5-yl)-amide,

2-Benzyl-5-tert-butyl-2H-pyrazole-3-carboxylic acid (2-methyl-1,3-benzothiazol-5-yl)-amide,

3-Fluoro-4-trifluoromethyl-N-(2-trifluoromethyl-1H-benzimidazol-5-yl)-benzamide,

2-Fluoro-5-trifluoromethyl-N-(2-trifluoromethyl-1H-benzimidazol-5-yl)-benzamide,

4-Chloro-N-(2-methyl-1,3-benzothiazol-5-yl)-benzamide,

1-Phenyl-5-trifluoromethyl-1H-pyrazole-3-carboxylic acid (2-methyl-1,3-benzothiazol-5-yl)-amide,

1-Phenyl-5-propyl-1H-pyrazole-4-carboxylic acid (2-methyl-1,3-benzothiazol-5-yl)-amide,

2,3-Difluoro-N-(2-methyl-1,3-benzothiazol-5-yl)-4-trifluoromethyl-benzamide,

3-Fluoro-4-methyl-N-(2-methyl-1,3-benzothiazol-5-yl)-benzamide,

4-tert-Butyl-N-(2-methyl-1,3-benzothiazol-5-yl)-benzamide,

4-Ethyl-N-(2-methyl-1,3-benzothiazol-5-yl)-benzamide,

4-tert-Butyl-N-(2-methyl-benzooxazol-5-yl)-benzamide,

Biphenyl-4-carboxylic acid (2-methyl-1,3-benzothiazol-5-yl)-amide,

3-Bromo-thiophene-2-carboxylic acid (2-methyl-1,3-benzothiazol-5-yl)-amide,

4-Bromo-2-methyl-N-(2-methyl-1,3-benzothiazol-5-yl)-benzamide,

4-tert-Butoxy-N-(2-methyl-1,3-benzothiazol-5-yl)-benzamide,

2-Chloro-3,4-dimethoxy-N-(2-methyl-1,3-benzothiazol-5-yl)-benzamide,

4-Iodo-N-(2-methyl-1,3-benzothiazol-5-yl)-benzamide,

4-Amino-N-(2-methyl-1,3-benzothiazol-5-yl)-3-nitro-benzamide,

N-(2-Methyl-1,3-benzothiazol-5-yl)-4-vinyl-benzamide,

4-Ethoxy-N-(2-methyl-1,3-benzothiazol-5-yl)-benzamide,

4-Ethylsulfanyl-N-(2-methyl-1,3-benzothiazol-5-yl)-benzamide,

4-Dimethylamino-naphthalene-1-carboxylic acid (2-methyl-1,3-benzothiazol-5-yl)-amide,

2-Fluoro-6-iodo-N-(2-methyl-1,3-benzothiazol-5-yl)-benzamide,

4-Ethoxymethyl-N-(2-methyl-1,3-benzothiazol-5-yl)-benzamide,

N-(2-Methyl-1,3-benzothiazol-5-yl)-4-trifluoromethoxy-benzamide, and

4-Chloro-3-fluoro-N-(2-methyl-1,3-benzothiazol-5-yl)-benzamide,

or salts, solvates or solvated salts thereof.

A further embodiment of the invention relates to compounds selected from the group consisting of

4-tert-Butyl-N-(2-formyl-1,3-benzothiazol-5-yl)-benzamide,

4-tert-Butyl-N-(2-hydroxymethyl- 1,3-benzothiazol-5-yl)-benzamide,

5-(4-tert-butylbenzoylamino)-1,3-benzothiazol-2-ylcarboxylic acid, and

4-tert-Butyl-N-(2-methoxycarbonyl-1,3-benzothiazol-5-yl)-benzamide,

or salts, solvates or solvated salts thereof.

Yet another embodiment of the invention relates to compounds selected from the group consisting of

4-tert-Butoxy-N-[2-(hydroxymethyl)-1,3-benzothiazol-5-yl]benzamide,

5 4-Bromo-N-[2-(hydroxymethyl)-1,3-benzothiazol-5-yl]benzamide,

N-[2-(Hydroxymethyl)-1,3-benzothiazol-5-yl]4-iodobenzamide,

N-[2-(Hydroxymethyl)-1,3-benzothiazol-5-yl]-4-morpholin4-ylbenzamide,

N-{2-[(Allyloxy)methyl]-1,3-benzothiazol-5-yl}-4-morpholin4-ylbenzamide,

N-[2-(Hydroxymethyl)-1,3-benzothiazol-5-yl]-1-phenyl-5-propyl-1H-pyrazole4-carboxamide,

1-tert-Butyl-N-[2-(hydroxymethyl)-1,3-benzothiazol-5-yl]-3-methyl-1H-pyrazole-5-carboxamide,

4-(Ethoxymethyl)-N-[2-(hydroxymethyl)-1,3-benzothiazol-5-yl]benzamide,

N-[2-(Hydroxymethyl)-1,3-benzothiazol-5-yl]-1-phenyl-1H-pyrazole-5-carboxamide,

4-Bromo-N-[2-(hydroxymethyl)-1,3-benzothiazol-5-yl]-2-methylbenzamide,

4-tert-Butoxy-N-(2-methyl-1,3-benzoxazol-5-yl) benzamide,

N-(4-Bromo-2-methyl-1,3-benzothiazol-5-yl)-4-tert-butylbenzamide,

4-tert-Butyl-N-(4,7-dibromo-2-methyl-1,3-benzothiazol-5-yl)benzamide,

N-[2-(hydroxymethyl)-1,3-benzothiazol-5-yl]-1-phenyl-5-(trifluoromethyl)-1H-pyrazole-4-carboxamide,

4-Iodo-N-(2-methyl-5-benzothiazolyl)benzamide,

4-(tert-Butoxymethyl)-N-[2-(hydroxymethyl)-1,3-benzothiazol-5-yl]benzamide,

N-(1,2-Dimethyl-1H-benzimidazol-5-yl)4-iodobenzamide,

4-tert-Butyl-N-(2-{[(2-methoxypyridin-3-yl)amino]methyl}-1,3-benzothiazol-5-yl)benzamide,

4-tert-Butyl-N-[2-(1-hydroxyethyl)-1,3-benzothiazol-5-yl)benzamide,

4-tert-Butyl-N- (2-((1H-pyrazol-3-ylamino)methyl]-1,3-benzothiazol-5-yl}benzamide,

4-(1,1-Dimethylethyl)-N-[2-[[(4-nitrophenyl)amino]methyl]-5-benzothiazolyl]-benzamide,

N-[2-(Aminomethyl)-1,3-benzothiazol-5-yl]-4-tert-butylbenzamide,

4-tert-Butyl-N-(2-{[(methylsulfonyl)amino]methyl}-1,3-benzothiazol-5-yl)benzamide,

N-{2-[(Acetylamino)methyl]-1,3-benzothiazol-5-yl 1-4-tert-butylbenzamide,

5-[(4-tert-Butylbenzoyl)amino]-1,3-benzothiazole-2-carboxamide,

N-1,3-Benzothiazol-5-yl-4-tert-butylbenzamide,

4-Chloro-N-[2-(hydroxymethyl)-1,3-benzothiazol-5-yl]benzamide,

1-(4-chlorophenyl)-N-[2-(hydroxymethyl)-1,3-benzothiazol-5-yl]-5-propyl-1H-pyrazole4-carboxamide,

1-(4-chlorophenyl)-N-[2-(hydroxymethyl)-1,3-benzothiazol-5-yl]-5-(trifluoromethyl)-1H-pyrazole4-carboxamide,

N-(2,4-dimethyl-1,3-benzothiazol-5-yl)4-(1-hydroxy-1-methylethyl)benzamide,

4-(Hydroxymethyl)-N-[2-(hydroxymethyl)-1,3-benzothiazol-5-yl]benzamid,e and

4-tert-butyl-N-(4-cyano-2-methyl-1,3-benzothiazol-5-yl)benzamide,

or salts, solvates or solvated salts thereof.

One embodiment of the invention relates to compounds selected from the group consisting of

N-[2-(Hydroxymethyl)-1,3-benzothiazol-5-yl]4-[2,2,2-trifluoro-1-hydroxy-1-(trifluoromethyl)ethyl]benzamide,

N-[2-(Hydroxymethyl)-1,3-benzothiazol-5-yl]4-isopropoxybenzamide,

4-Bromo-2-chloro-N-[2-(hydroxymethyl)-1,3-benzothiazol-5-yl]benzamide,

4-Bromo-2-fluoro-N-[2-(hydroxymethyl)-1,3-benzothiazol-5-yl]benzamide,

N-[2-(Hydroxymethyl)-1,3-benzothiazol-5-yl]-4-(morpholin-4-ylmethyl)benzamide,

3-Fluoro-N-[2-(hydroxymethyl)-1,3-benzothiazol-5-yl]4-(trifluoromethyl)benzamide,

4-tert-butoxy-N-[4-chloro-2-(hydroxymethyl)-1,3-benzothiazol-5-yl]benzamide,

4-(tert-Butoxymethyl)-N-[4-chloro-2-(hydroxymethyl)-1,3-benzothiazol-5-yl]benzamide, and

4-tert-butyl-N-[2-(hydroxymethyl)-1,3-benzoxazol-5-yl]benzamide,

or salts, solvates or solvated salts thereof.

Listed below are definitions of various terms used in the specification and claims to describe the present invention.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the other definitions for that group.

For the avoidance of doubt it is to be understood that in this specification ‘C₁₋₆’ means a carbon group having 1, 2, 3, 4, 5 or 6 carbon atoms.

In this specification, unless stated otherwise, the term “alkyl” includes both straight and branched chain alkyl groups and may be, but are not limited to methyl, ethyl, n-propyl, i-propyl, n-butyl, i-butyl, s-butyl, t-butyl, n-pentyl, i-pentyl, t-pentyl, neo-pentyl, n-hexyl or i-hexyl, t-hexyl. The term C₁₋₃ alkyl having 1 to 3 carbon atoms and may be methyl, ethyl, n-propyl, i-propyl or tert-butyl.

The term ‘C₀’ means a bond or does not excist. For example when R⁴ is C₀alkyl, R⁴ does not excist and “arylC₀alkyl” is equivalent with “aryl”, “C₂aklylOC₀alkyl” is equivalent with “C₂alkylO”.

In this specification, unless stated otherwise, the term “alkenyl” includes both straight and branched chain alkenyl groups. The term “C₂₋₆alkenyl” having 2 to 6 carbon atoms and one or two double bonds, may be, but is not limited to vinyl, allyl, propenyl, butenyl, crotyl, pentenyl, or hexenyl, and a butenyl group may for example be buten-2-yl, buten-3-yl or buten-4-yl.

In this specification, unless stated otherwise, the term “alkynyl” includes both straight and branched chain alkynyl groups. The term “C₂₋₆alkynyl” having 2 to 6 carbon atoms and one or two trippel bonds, may be, but is not limited to etynyl, propargyl, pentynyl or hexynyl and a butynyl group may for example be butyn-3-yl or butyn-4-yl.

In this specification, unless stated otherwise, the term “cycloalkyl” refers to an optionally substituted, saturated cyclic hydrocarbon ring system. The term “C₃₋₇cycloalkyl” may be cyclopropyl, cyclobutyl, cyclopentyl, cyclohexyl or cycloheptyl.

The term “heterocycloalkyl” denotes a 3- to 7-membered, non-aromatic, partially or completely saturated hydrocarbon group, which contains one ring and at least one heteroatom. Examples of said heterocycle include, but are not limited to pyridyl, pyrrolyl, furyl, thienyl, imidazolyl, oxazolyl, isoxazolyl, thiazolyl, pyrazolyl, benzofuryl, indolyl, isoindolyl, benzimidazolyl, pyridazinyl, pyrimidinyl, pyrazinyl, tetrazolyl, triazolyl, pyrrolidinyl, pyrrolidonyl, piperidinyl, piperazinyl, morpholinyl, oxazolyl, 2-oxazolidonyl or tetrahydrofuranyl.

In this specification, unless stated otherwise, the term “aryl” refer to an optionally substituted monocyclic or bicyclic hydrocarbon unsaturated aromatic ring system. Examples of “aryl” may be, but are not limited to phenyl and naphthyl.

In this specification, unless stated otherwise, the term “heteroaryl” refer to an optionally substituted monocyclic or bicyclic unsaturated aromatic ring system containing at least one heteroatom selected independently form N, O or S. Examples of “heteroaryl” may be, but are not limited to pyridyl, pyrrolyl, furyl, thienyl, imidazolyl, oxazolyl, isoxazolyl, thiazolyl, pyrazolyl, benzofuryl, indolyl, isoindolyl, benzimidazolyl, pyridazinyl, pyrimidinyl, pyrazinyl, tetrazolyl, triazolyl and oxazolyl.

In this specification, unless stated otherwise, the term “arylalkyl” and “heteroarylalkyl” refer to a substituent that is attached via the alkyl group to an aryl or heteroaryl group.

In this specification, unless stated otherwise, the term “halo” and “halogen” may be fluoro, iodo, chloro or bromo.

In this specification, unless stated otherwise, the term “haloalkyl” means an alkyl group as defined above, which is substituted with halo as defined above. The term “C₁₋₆haloalkyl” may include, but is not limited to fluoromethyl, difluoromethyl, trifluoromethyl, fluoroethyl, difluoroethyl or bromopropyl. The term “C₁₋₆haloalkylO” may include, but is not limited to fluoromethoxy, difluoromethoxy, trifluoromethoxy, fluoroethoxy or difluoroethoxy.

The present invention relates to the compounds of formula I as hereinbefore defined as well as to the salts, solvates or solvated salts thereof. Salts for use in pharmaceutical compositions will be pharmaceutically acceptable salts, but other salts may be useful in the production of the compounds of formula I.

A suitable pharmaceutically acceptable salt of the compounds of the invention is, for example, an acid-addition salt, for example an inorganic or organic acid. In addition, a suitable pharmaceutically acceptable salt of the compounds of the invention is an alkali metal salt, an alkaline earth metal salt or a salt with an organic base.

Other pharmaceutically acceptable salts and methods of preparing these salts may be found in, for example, Remington's Pharmaceutical Sciences (18^{th} Edition, Mack Publishing Co.).

Some compounds of formula I may have chiral centres and/or geometric isomeric centres (E- and Z- isomers), and it is to be understood that the invention encompasses all such optical, diastereoisomeric and geometric isomers.

The invention also relates to any and all tautomeric forms of the compounds of formula I.

Methods of Preparation

Another aspect of the present invention provides processes for preparing compounds of formula I, or salts, solvates or solvated salts thereof.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). References and descriptions of other suitable reactions are described in textbooks of organic chemistry, for example, “Advanced Organic Chemistry”, March, 4^{th} ed. McGraw Hill (1992) or, “Organic Synthesis”, Smith, McGraw Hill, (1994). For representative examples of heterocyclic chemistry see for example “Heterocyclic Chemistry”, J. A. Joule, K. Mills, G. F. Smith, 3^{rd} ed. Chapman and Hall (1995), p. 189-224 and “Heterocyclic Chemistry”, T. L. Gilchrist, 2^{nd} ed. Longman Scientific and Technical (1992), p. 248-282.

The term “room temperature” and “ambient temperature” shall mean, unless otherwise specified, a temperature between 16 and 25° C.

One embodiment of the invention relates to processes for the preparation of the compound of formula I, wherein R¹ to R⁸, unless otherwise specified, are defined as in formula I, comprising;

a) reaction of an aromatic amine of formula (II) with a properly substituted acyl chloride (III) optionally in the presence of a base:

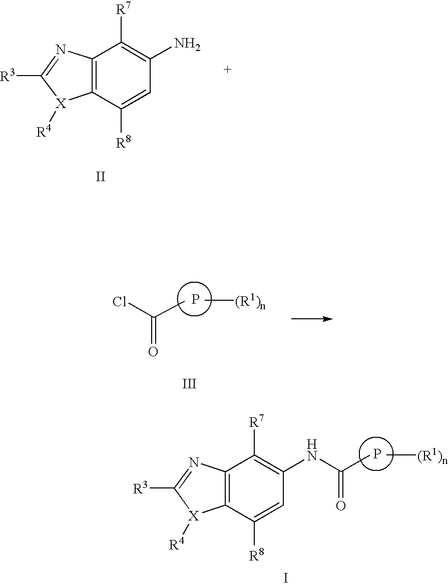

This reaction may be performed in any manner known to the skilled person in the art. Suitable solvents to be used for this reaction may be halogenated hydrocarbons such as chloroform, dichloromethane and dichloroethane or aromatic and heteroaromatic compounds such as benzene, toluene, xylene, pyridine and lutidine or ethers such as ethyl ether, tetrahydrofuran and dioxan or any mixtures thereof. Catalysts such as heteroaromatic bases like pyridine and lutidine or tertiary amines like triethylamine, N-methylmorpholine and ethyl diisopropylamine or polymer bound tertiary amines like N,N-(diisopropyl)aminomethylpolystyrene resin may be used as well. The temperature may be between 40 and 40° C. and the reaction time may be between 0.5 and 30 h.

b) reaction of an aromatic amine of formula (II) with a properly substituted acid (IV) in the presence of a coupling agent (activator) like for example 1-[3-(dimethylamino)propyl]-3-ethylcarbodiimide hydrochloride.

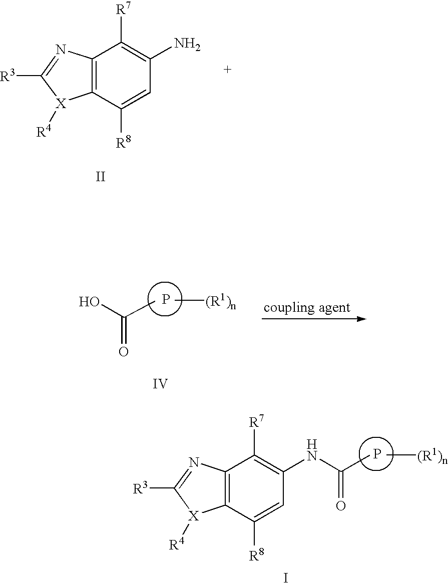

Suitable solvents to be used for this reaction may be tertiary amides such as dimethylformamide and dimethylacetamide, halogenated hydrocarbons such as chloroform, dichloromethane and dichloroethane or aromatic and heteroaromatic compounds such as benzene, toluene, xylene, pyridine and lutidine or ethers such as ethyl ether, tetrahydrofuran and dioxan or any mixtures thereof. Catalysts such as heteroaromatic bases like pyridine and lutidine or tertiary amines like triethylamine, N-methylmorpholine and ethyl diisopropylamine may be used as well. The temperature may be between 10 and 60° C. and the reaction time may be between 3 and 30 h.

c) reaction of an hydroxymethyl derivative Ia with methanesulfonyl chloride followed by treatment with ammonia.

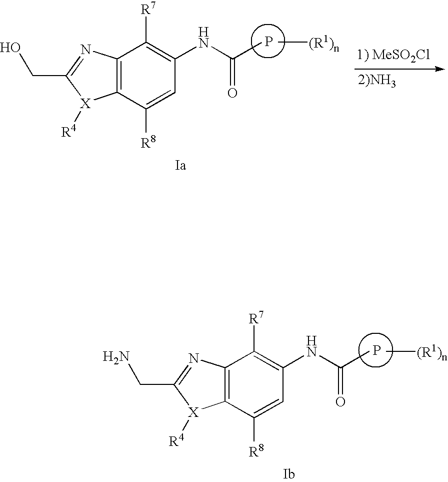

The mesylation step is carried out using halogenated hydrocarbons such as chloroform, dichloromethane and dichloroethane as a solvent and a tertiary amine like triethylamine, N-methylmorpholine and ethyl diisopropylamine as a base in a temperature range between -20 and 30° C. The amination step step is carried out using a solution of ammonia in an alcohol like ethanol or in an aprotic solvent like dioxane or in water.

d) reaction of an aminomethyl derivative Ib with an acyl chloride or a sulfonyl chloride

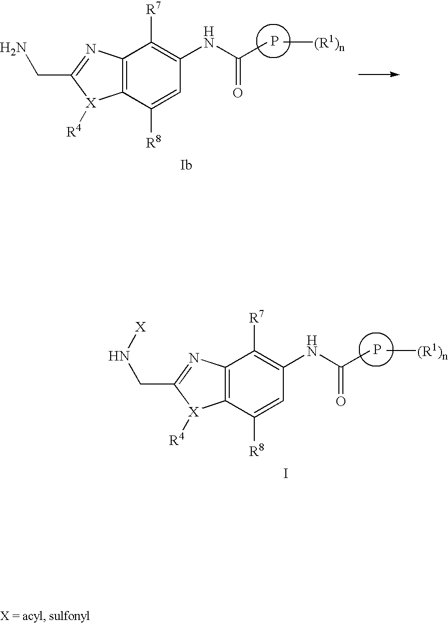

The reaction conditions are similar to the ones described for the mesylation step in part c).

e) oxidation of the aldehyde Ic to the corresponding carbonic acid Id

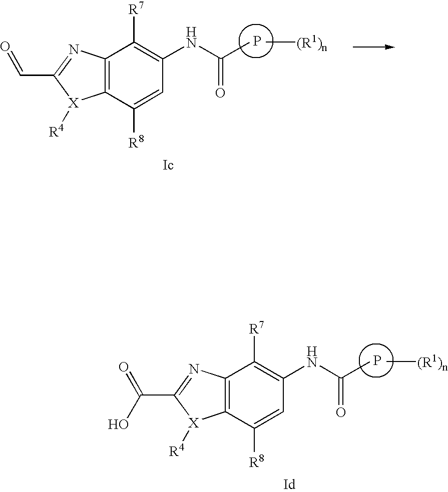

For the oxidation purpose a mixture of sodium chlorite and sulfamic acid in water may be employed

f) decarboxylation of the carbonic acid Id

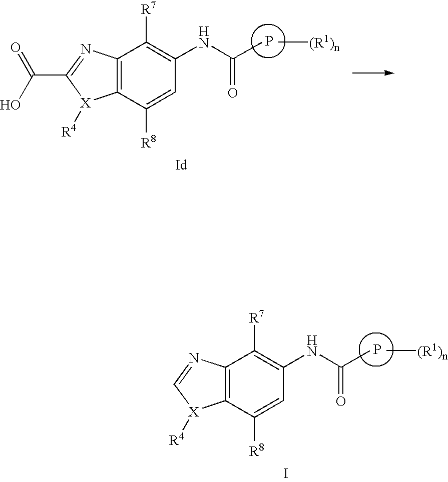

g) esterification of the carbonic acid Id

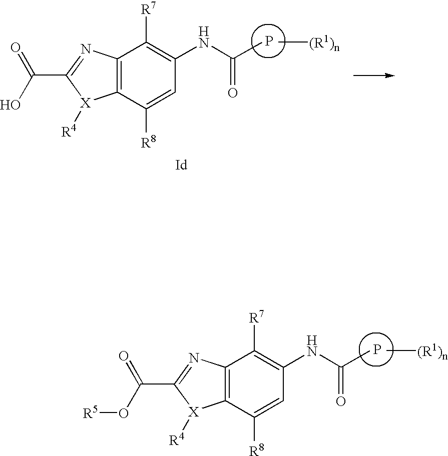

h) amidation of the carbonic acid Id

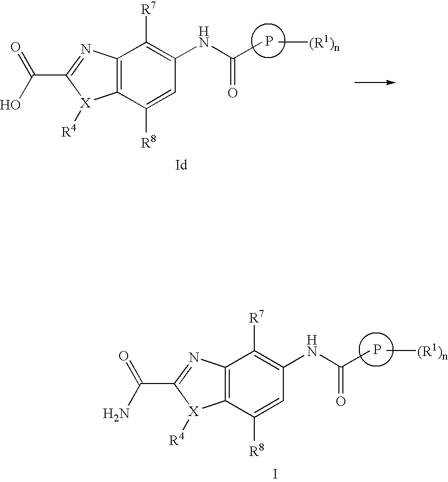

i) reduction of the aldehyde Ic to a corresponding primary alcohol

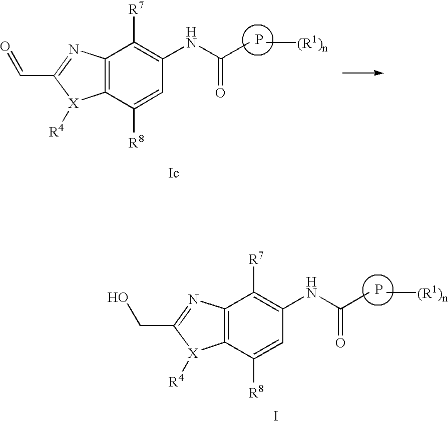

As a suitable reductive agent sodium borohydride may be used in a solvent like methanol or another alcohol or its mixture with water in a temperature range between -10 and 40° C.

j) treatment of the aldehyde Ic with organometallic reagent leading to secondary alcohols

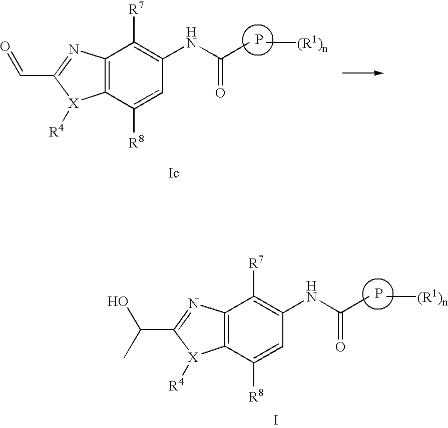

Organometalic reagent may be a magnesium derivatives like methylmagnesium bromide or organolithium compound like methyllithium and a suitable solvent may be chosen from a range of aprotic inert solvents like diethyl ether, tetrahydrofuran, benzene, etc.

k) reductive amination of the aldehyde Ic

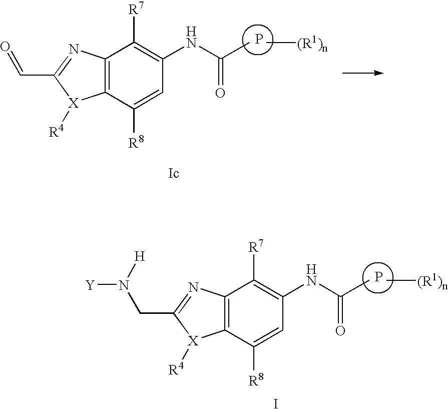

Y is aryl, heteroaryl in process i) any primary amine may be used together with an appropriate reductive agent for example decaborane or sodium cyanoborohydride. Both protic and aprotic solvents, for example, alcohols, water, terahydrofuran and mixtures thereof are suitable and the temperature range is between 0 and 40° C.

l) reduction of the aldehyde Ie to a corresponding primary alcohol as in part i)

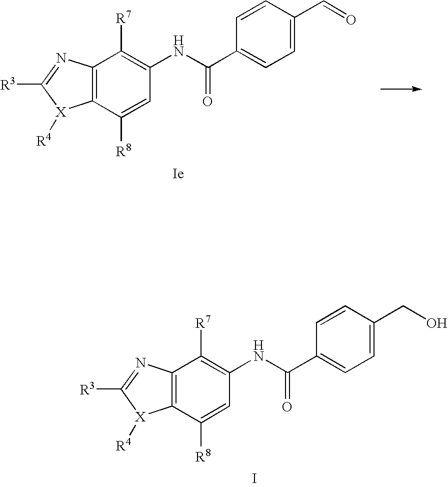

m) treatment of the methyl ester If with organometallic reagent leading to tertiary alcohols in a similar way to the process described in part j)

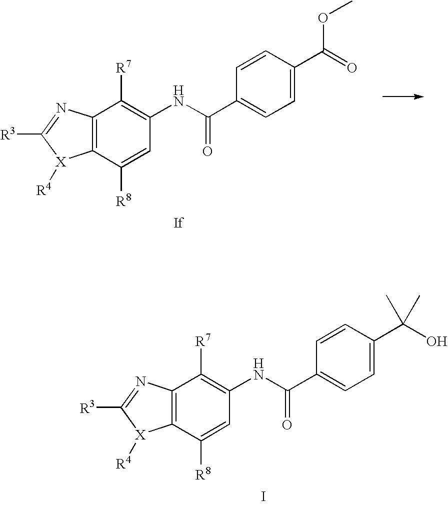

n) reaction of the bromo derivative Ig with a cyanation reagent

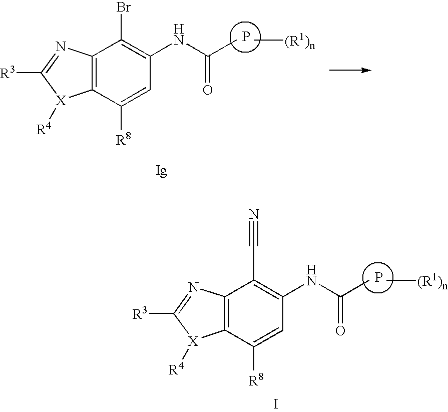

As a cyanation reagent copper (I) cyanide may be used in an aprotic polar solvent having high boiling point, like dimethyl formamide, at elevated temperature in a range between 150 and 270° C.

o) oxidation of the 2-methyl derivative Ih and subsequent reduction of the intermediary aldehyde to the 2-hydroxymethyl derivative Ii

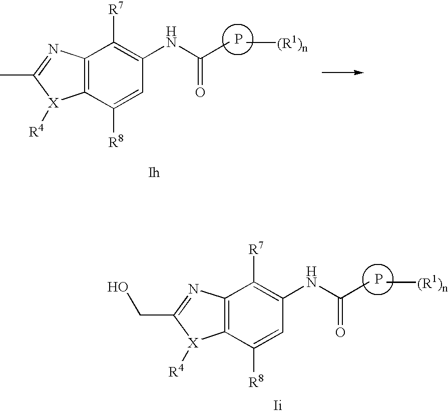

The oxidation step is accomplished by using an appropriate oxidative reagent for example, magnesium dioxide, chromium trioxide or selenium dioxide. Suitable solvents to be used for this reaction may be ketones such as acetone and butan-2-one, or halogenated hydrocarbons such as chloroform, dichloromethane and dichloroethane or any mixtures thereof. The temperature may be between 0 and 80° C. and the reaction time may be between 3 and 50 h. The subsequent reduction is typically carried out using sodium borohydride in methanol.

### Abbreviations

alloc allyloxycarbonyl
DCE dichloroethane
DCM dichloromethane
DMAP dimethylaminopyridine
EDC 1-(3-dimethylaminopropyl)-3-ethylcarbodiimide hydrochloride
HATU O-(7-azabenzotriazol-1-yl)-N,N,N′,N′-tetramethyluronium hexafluorophosphate
HPLC high performance liquid chromatography
LC liquid chromatography
MsCl methanesulfonyl chloride
MS mass spectometry
ret. time retention time
TFA trifluroacetic acid

A further embodiment of the invention relates to compounds

allyl (5-amino-1,3-benzothiazol-2-yl)methyl carbonate,

4-tert-Butyl-N-(2-formyl-1,3-benzothiazol-5-yl)-benzamide, and

4-Bromo-2-methyl-benzothiazol-5-ylamine, and

4-chloro-2-methyl-benzothiazole-5-ylamine.

Another embodiment relates to the used of these compounds as intermediates in the preparation of the compound of formula I.

Pharmaceutical Composition

According to one embodiment of the present invention there is provided a pharmaceutical composition comprising as active ingredient a therapeutically effective amount of the compound of formula I, or salts, solvates or solvated salts thereof, in association with one or more pharmaceutically acceptable diluents, excipients and/or inert carriers.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration e.g. as an ointment, patch or cream or for rectal administration e.g. as a suppository.

In general the above compositions may be prepared in a conventional manner using one or more conventional excipients, pharmaceutical acceptable diluents and/or inert carriers. Suitable daily doses of the compounds of formula I in the treatment of a mammal, including man, are approximately 0.01 to 250 mg/kg bodyweight at peroral administration and about 0.001 to 250 mg/kg bodyweight at parenteral administration.

The typical daily dose of the active ingredient varies within a wide range and will depend on various factors such as the relevant indication, severity of the illness being treated, the route of administration, the age, weight and sex of the patient and the particular compound being used, and may be determined by a physician.

Examples of Pharmaceutical Composition

The following illustrate representative pharmaceutical dosage forms containing a compound of formula I, or salts, solvates or solvated salts thereof, (hereafter compound X), for preventive or therapeutic use in mammals:

| (a): Tablet | mg/tablet |
|---|---|
| Compound X | 100 |
| Lactose | 182.75 |
| Croscarmellose sodium | 12.0 |
| Maize starch paste (5% w/v paste) | 2.25 |
| Magnesium stearate | 3.0 |
| (b): Capsule | mg/capsule |
| Compound X | 10 |
| Lactose | 488.5 |
| Magnesium stearate | 1.5 |
| (c): Injection | (50 mg/ml) |
| Compound X | 5.0% w/v |
| 1M Sodium hydroxide solution | 15.0% v/v |
| 0.1M Hydrochloric acid | (to adjust pH to 7.6) |
| Polyethylene glycol 400 | 4.5% w/v |
| Water for injection | up to 100% |

The above compositions may be obtained by conventional procedures well known in the pharmaceutical art.

Medical Use

Surprisingly, it has been found that the compounds according to the present invention are useful in therapy. The compounds of formula I, or salts, solvates or solvated salts thereof, as well as their corresponding active metabolites, exhibit a high degree of potency and selectivity for individual vanilloid receptor 1 (VR1) groups. Accordingly, the compounds of the present invention are expected to be useful in the treatment of conditions associated with excitatory activation of vanilloid receptor 1 (VR1).

The compounds may be used to produce an inhibitory effect of VR1 in mammals, including man.

VR1 are highly expressed the peripheral nervous system and in other tissues. Thus, it is expected that the compounds of the invention are well suited for the treatment of VR1 mediated disorders.

The compounds of formula I are expected to be suitable for the treatment of acute and chronic pain, acute and chronic neuropathic pain and acute and chronic inflammatory pain.

Examples of such disorder may be selected from the group comprising arthritis, fibromyalgia, low back pain, post-operative pain, visceral pains like chronic pelvic pain, cystitis, including interstitial cystitis, bowel syndrome (IBS), pancreatitis, ischeamic, sciatia, diabetic neuropathy, multiple sclerosis, HIV neuropathy, asthma, cough and inflammatory bowel disease (IBD).

Further relevant disorders may be selected from the group comprising gastro-esophageal reflux disease (GERD), psoriasis, cancer, emesis, urinary incontinence and hyperactive bladder.

Other relevant disorders are related to respiratory diseases and may be selected from the group comprising asthma, chronic obstructive lung disease and emphysema, lung fibrosis and interstitial lung disease.

The VR1 inhibitor(s) may be administrated by either an oral or inhaled route. The respiratory disease may be an acute and chronic illness and may be related to infection(s) and/or exposure to environmental pollution and/or irritants.

The compounds of formula I may also be used as antitoxin to treat (over-) exposure to VR1 activators like capsaicin, tear gas, acids or heat. Regarding heat, there is a potential use for VR1 antagonists in (sun-) burn induced pain, or inflammatory pain resulting from brun injuries.

The compounds may further be used for treatment of tolerance to VR1 activators. One embodiment of the invention relates to the use of the compounds of formula I as hereinbefore defined, in therapy.

Another embodiment of the invention relates to the use of the compounds of formula I as hereinbefore defined, for treatment of VR1 mediated disorders.

A further embodiment of the invention relates to the use of the compounds of formula I as hereinbefore defined, for treatment of acute and chronic pain disorders.

Yet another embodiment of the invention relates to the use of the compounds of formula I as hereinbefore defined, for treatment of acute and chronic neuropathic pain.

Yet a further embodiment of the invention relates to the use of the compounds of formula I as hereinbefore defined, for treatment of acute and chronic inflammatory pain.

One embodiment of the invention relates to the use of the compounds of formula I as hereinbefore defined, for treatment of arthritis, fibromyalgia, low back pain, post-operative pain, visceral pains like chronic pelvic pain, cystitis, IBS, pancreatitis or ischeamic.

Another embodiment of the invention relates to the use of the compounds of formula I as hereinbefore defined, for treatment of sciatia, diabetic neuropathy, multiple sclerosis or HIV neuropathy.

A further embodiment of the invention relates to the use of the compounds of formula I as hereinbefore defined, for treatment of asthma, cough, IBD, psoriasis, gastro-esophageal reflux disease (GERD), psoriasis, cancer, emesis, urinary incontinence or hyperactive bladder.

Yet another embodiment of the invention relates to the use of the compounds of formula I as hereinbefore defined, for treatment of interstitial cystitis and pain related to interstitial cystitis.

Yet a further embodiment of the invention relates to the use of the compound of formula I as hereinbefore defined, for the treatment of respiratory diseases selected from the group comprising asthma, chronic obstructive lung disease and emphysema, lung fibrosis and interstitial lung disease.

One embodiment of the invention relates to the use of the compound of formula I as hereinbefore defined, in the manufacture of a medicament for treatment of VR1 mediated disorders and for treatment of acute and chronic pain disorders, acute and chronic neuropathic pain and acute and chronic inflammatory pain and any other disorder mentioned above.

Another embodiment of the invention relates to a method of treatment of VR1 mediated disorders and acute and chronic pain disorders, acute and chronic neuropathic pain and acute and chronic inflammatory pain, and respiratory diseases, and any other disorder mentioned above, comprising administrering to a mammal, including man in need of such treatment, a therapeutically effective amount of the compounds of formula I, as hereinbefore defined.

A further embodiment of the invention relates to a pharmaceutical composition comprising a compound of formula I as hereinbefore defined, for use in treatment of VR1 mediated disorders and for treatment of acute and chronic pain disorders, acute and chronic neuropathic pain and acute and chronic inflammatory pain and any other disorder mentioned above.

In the context of the present specification, the term “therapy” and “treatment” includes prevention and prophylaxis, unless there are specific indications to the contrary. The terms “treat”,“therapeutic” and “therapeutically” should be construed accordingly.

In this specification, unless stated otherwise, the term “inhibitor” and “antagonist” mean a compound that by any means, partly or completely, blocks the transduction pathway leading to the production of a response by the ligand.

The term “disorder”, unless stated otherwise, means any condition and disease associated with vanilloid receptor activity.

Non-Medical Use

In addition to their use in therapeutic medicine, the compounds of formula I, or salts, solvates or solvated salts thereof, are also useful as pharmacological tools in the development and standardisation of in vitro and in vivo test systems for the evaluation of the effects of inhibitors of VR1 related activity in laboratory animals such as cats, dogs, rabbits, monkeys, rats and mice, as part of the search for new therapeutics agents.

### EXAMPLES

The invention will now be illustrated by the following non-limiting examples.

General Methods

All starting materials are commercially available or described in the literature. The ¹H NMR spectra were recorded on Brucker at 400 MHz. The mass spectra were recorded utilising electrospray (LC-MS; LC:Waters 2790, column XTerra MS C₈ 2.5 μm 2.1×30 mm, buffer gradient H₂O+0.1% TFA:CH₃CN+0.04% TFA, MS: micromass ZMD//ammonium acetate buffer) ionisation techniques.

Synthesis of Aromatic Amines as Starting Materials employed in amide bond-forming reactions in examples 1-17.

### Allyl (5-amino-1,3-benzothiazol-2-yl)methyl carbonate

### A. tert-Butyl (2-methyl-1,3-benzothiazol-5-yl)carbamate

A mixture of Et₃N (100 mL), di-tert-butyl dicarbonate (58.3 g, 267 mmol) and 5-amino-2-methylbenzothiazole (22.0 g, 134 mmol) in MeOH (300 mL) was stirred at 65° C. for 2 hours and room temperature for 18 hours. The mixture was concentrated under reduced pressure, and the residue was diluted with DCM and washed with a 1M solution of HCl. The organic phase was dried with Na₂SO₄, filtered and evaporated under reduced pressure to yield the carbamate derivative. R_{f}=0.45 (hexanes:EtOAc, 1:1); MS [M+] calc. 264.0, found 264.9.

### B. tert-Butyl [2-(hydroxymethyl)-1,3-benzothiazol-5-yl]carbamate

SeO₂ (45.0 g, 402 mmol) was ground to a fine powder and added to a solution of the carbamate in dioxane (300 mL). The mixture was kept under a N₂ atmosphere and heated to 70° C. for 18 hours with vigorous stirring. The mixture was quickly filtered and the solid was washed with hot dioxane. The filtrate was concentrated under reduced pressure to yield the aldehyde. R_{f}=0.56 (hexanes:EtOAc, 1:1). The crude aldehyde was dissolved in MeOH (300 mL) and NaBH₄ (15.21 g, 402 mmol) was added portion-wise. The mixture was stirred for 2 hours and then diluted with 1M NaOH. The mixture was evaporated to dryness, dissolved in DCM, washed with a saturated solution of NaHCO₃, dried with Na₂SO₄, filtered and concentrated under reduced pressure. R_{f}=0.09 (hexanes:EtOAc, 2:1); MS [M+] calc. 280.0 found 280.9.

### C. Allyl {5-[(tert-butoxycarbonyl)amino]-1,3-benzothiazol-2-yl)methyl carbonate

The primary alcohol was dissolved in DCM (300 mL), and allylchloroformate (16.2 mg, 134 mmol) was added followed by DMAP (14.2 g, 140 mmol). The mixture was stirred for 3 hours, and the solvent was evaporated. MS [M+] calc. 364.0 found 364.9.

### D. Allyl (5-amino-1,3-benzothiazol-2-yl)methyl carbonate

The alloc-protected derivative was dissolved in DCM (300 mL), and TFA (100 mL) was added. The mixture was stirred for 18 hours, and then concentrated under reduced pressure. The product was purified by flash chromatography on silica gel eluting with mixtures of heptane and EtOAc (4:1, 7:3 and 1:1) to yield an off-white powder (6.6 g, 25 mmol). ¹H NMR (400 MHz, CHLOROFORM-D) δ ppm 4.71 (d, J=5.86 Hz, 2 H) 5.30 (dd, J=10.35, 1.17 Hz, 1 H) 5.37 (q, J=3.0, 1.50 Hz, 1 H) 5.42 (q, J=3.0, 1.50 Hz, 1 H) 5.51 (s, 2 H)5.95 (m, 1 H) 7.10 (s, 1 1 H) 7.63 (s, 1 H) 7.70 (d, J=8.01 Hz, 1 H); ¹³C NMR (101 MHz, DMSO-D6) δ ppm 65.9, 68.5, 110.7, 117.5, 118.7, 122.9, 126.9, 131.9, 140.5, 153.4, 153.9, 166.7; MS [M+] calc. 264.0 found 264.8.

### Allyl (5-amino-4-chloro-1,3.benzothiazol-2-yl)methyl carbonate

Allyl (5-amino-1,3-benzothiazol-2-yl)methyl carbonate (500 mg, 1.89 mmol) was dissolved in DCM (19.0 mL) and N-chlorosuccinimide (253 mg, 1.89 mmol) was added. The mixture was stirred at ambient temperature until the reaction appeared complete by LC-MS. The solution was concentrated under reduced pressure and purified by flash chromatography using mixtures of hexanes and EtOAc (4:1, 2:1) as an eluent to yield the title product (429 mg, 1.44 mmol, 76%). ¹H NMR (400 MHz, chloroform-D) δ ppm 2.77 (s, 2H) 4.71 (dt, J=5.86, 2.73, 1.37 Hz, 2 H) 5.27-5.46 (m, 2 H) 5.57 (s, 2 H) 5.89-6.05 (m, 1 H) 6.92 (d, J=8.59 Hz, 1 H) 7.55 (d, J=8.59 Hz, 1 H).

### 4-Bromo-2-methyl-1,3-benzothiazol-5-ylamine and 4,6-Dibromo-2-methyl-benzothiazol-5-ylanine

5-Amino-2-methylbenzothiazole (2.45 g, 14.9 mmol) and Br₂ (2.38 g, 14.9 mmol) were mixed in CHCl₃ (60.0 mL) and stirred for 45 minutes. 28% NH₄OH (20.0 mL) was added, and the aqueous phase was extracted with DCM. The combined organic phases were dried with MgSO₄, filtered and evaporated. The products were separated from by flash chromatography on silica gel eluting with mixtures of hexanes and EtOAc (4:1) to yield 4-bromo-2-methyl-1,3-benzothiazol-5-ylamine: LC ret. time 1.13 minutes (Column: Phenomonex Polar, Gradient: 10-95% B, Flow rate: 1.75 mL/min, Column temperature: 40° C., Mobile phase: A-0.1% TFA in H₂O, B-0.1% TFA in MeCN), MS [M+] calcd. 242.0, found 242.0; and 4,6-dibromo-2-methyl-benzothiazol-5-ylamine: LC ret. time 1.64 minutes MS [M+] calcd. 322.0, found 322.0

### 5-Amino-1,3-benzothiazole-2-carbaldehyde

Manganese dioxide (10 mmol) was added to a solution of 5-amino-2-methylbenzothiazole (2 mmol) in acetone (20 mL ). The mixture was refluxed for 24 h. After cooling to ambient temperature, the mixture was filtered and concentrated in vacuo to afford crude 5-amino-2-formylbenzothiazole as a yellow oil which was used for the next step without further purification. MS [M+] calc. 178.2, found 179

### Example 1

### 4-tert-Butoxy-N-[2-(hydroxymethyl)-1,3-benzothiazol-5-yl]benzamide

A. Synthesis of the O-Alloc Protected Derivative

Allyl (5-amino-1,3-benzothiazol-2-yl)methyl carbonate (see above) (97.0 mg, 0.370 mmol) and 4-tert-butoxybenzoic acid (71.0 mg, 0.370 mmol) were mixed in a mixture of DCM (5.00 mL) and DMF (5.00 mL) with EDC (220 mg, 1.15 mmol) and DMAP (236 mg, 1.15 mmol). ). The mixture was stirred for 18 hours, and the solvents were evaporated. The residue was dissolved in DCM and washed with a saturated solution of NaHCO₃. The mixture was dried with Na₂SO₄, filtered and concentrated under reduced pressure. The crude product was purified by Gilson reverse phase HPLC (Luna 15 u, C18 (2) 250 mm×21.2 mm), eluting with mixtures of H₂O and MeCN with 0.1% TFA the O-alloc protected derivative of the title compound: MS [M+] calc. 440.0 found 440.9.

B. Deprotection

The product obtained in Part A was treated with a solution of Pd(OAc)₂ (10.0 mg), PPh₃ (20.0 mg) and Et₃SiH (176 mg, 1.52 mmol, 0.240 mL) in a mixture of THF (4.00 mL) and DMF (4.00 mL). The mixture was stirred at room temperature until the reaction appeared complete by TLC analysis, and the solvents were evaporated. The crude product was purified by Gilson HPLC (Luna 15 u, C18 (2), 250 mm×21.2 mm) eluting with mixture of MeCN and H₂O containing 1% TFA to yield the title product. ¹H NMR (400 MHz, methanol-D4) δ ppm 1.42 (s, 9 H) 4.95 (s, 2 H) 7.12 (m, 2 H) 7.71 (dd, J=8.79, 1.17 Hz, 1 H) 7.91 (m, 2 H) 7.96 (d, J=8.79 Hz, 1 H) 8.40 (s, 1 H); MS [M+H] calc. 357.1 found 10 357.0; Anal. found C 64.61% H 5.58% N 6.65%.

### Example 2

### 4-Bromo-N-[2-(hydroxymethyl)-1,3-benzothiazol-5-yl]benzamide

4-Bromobenzoylchloride (0.4 mmol) was dissolved in DCM and DMAP (0.4 mmol) was added. The mixture was stirred for 10 minutes and then allyl (5-amino-1,3-benzothiazol-2-yl)methyl carbonate (100 mg, 0.38 mmol) was added. The mixture was stirred until the reaction appeared complete by TLC analysis and NaOH (1M) was added. The aqueous phase was extracted with DCM. The organic phases were collected, dried with Na₂SO₄, filtered and concentrated under reduced pressure. Purification by HPLC afforded the O-alloc protected derivative of the title compound: MS [M+1] calc. 448.0 found 448.4. Deprotection according to the procedure described in Example 1, part B afforded the title compound; δ ppm 4.79 (s, 2H) 7.55 (d, J=8.3 Hz, 3 H) 7.74 (d, J=8.4 Hz, 2 H) 7.8 (d, J=8.7 Hz, 1 H) 8.25 (s, 1 H); MS [M+H] calc. 363.2 found 363.0.

Compounds in the following examples were synthesized according to the amide bond-forming procedures described in the examples 1 or 2 starting from an appropriate aromatic amine, either commercially available or synthesized according to the procedures described above, and an appropriately substituted commercially available aromatic acid or an aromatic acyl chroride. Where appropriate the amide bond-forming procedures were followed by the deprotection as described in Example 1

| | | | MW | |
|---|---|---|---|---|
| Example | | MW | found | |
| number | Name | calcd | [M + 1] | ¹H NMR |
| 3 | N-[2-(Hydroxymethyl)- | 411.1 | 411.0 | (600MHz, methanol-D₄) δppm |
| | 1,3-benzothiazol-5-yl]-4- | | | 4.79(s, 2H) 7.55(d, |
| | iodobenzamide | | | J=8.3Hz, 3H) 7.74(d, J=8.4Hz, |
| | | | | 2H) 7.80(d, J=8.7Hz, 1H) |
| | | | | 8.25(s, 1H) |
| 4 | N-[2-(Hydroxymethyl)- | 370.1 | 370.0 | (400MHz, DMSO-D₆) δppm |
| | 1,3-benzothiazol-5-yl]-4- | | | 3.24(m, 4H), 3.73(m, 4H) |
| | morpholin-4-ylbenzamide | | | 4.83(d, J=6.05Hz, 2H) |
| | | | | 6.22(t, J=5.96Hz, 1H) 7.02(d, |
| | | | | J=9.18Hz, 2H) 7.76(dd, |
| | | | | J=8.69, 2.05Hz, 1H) 7.90(d, |
| | | | | J=9.18Hz, 2H) 7.98(d, |
| | | | | J=8.79Hz, 1H) 8.42(d, |
| | | | | J=1.95Hz, 1H) 10.13(s, 1H) |
| 5 | N-{2-[(Allyloxy)methyl]- | 410.2 | 410.0 | (400MHz, methanol-D₄) δppm |
| (no | 1,3-benzothiazol-5-yl}-4- | | | 3.09(m, 4H), 3.73(m, 4H) |
| deprotection | morpholin-4-ylbenzamide | | | 4.58(d, J=6.05Hz, 2H) |
| step | | | | 4.90(s, 2H) 5.16(m, 2H) |
| required) | | | | 5.99(m, 1H) 6.70(m, 2H) |
| | | | | 7.22(m, 3H) 7.62(d, J=1.95Hz, |
| | | | | 1H) 7.87(d, J=8.59Hz, 1H) |
| 6 | N-[2-(Hydroxymethyl)- | 393.1 | 392.9 | (400MHz, methanol-D₄) δppm |
| | 1,3-benzothiazol-5-yl]-1- | | | 3.09(m, 4H), 3.73(m, 4H) |
| | phenyl-5-propyl-1H- | | | 4.58(d, J=6.05Hz, 2H) |
| | pyrazole-4-carboxamide | | | 4.90(s, 2H) 5.16(m, 2H) |
| | | | | 5.99(m, 1H) 6.70(m, 2H) |
| | | | | 7.22(m, 3H) 7.62(d, J=1.95Hz, |
| | | | | 1H) 7.87(d, J=8.59Hz, 1H) |
| 7 | 1-tert-Butyl-N-[2- | 345.1 | 345.0 | (400MHz, methanol-D₄) δppm |
| | (hydroxymethyl)-1,3- | | | 1.71(s, 9H) 2.52(s, 3H) |
| | benzothiazol-5-yl]-3- | | | 4.95(s, 2H) 6.61(s, 1H) |
| | methyl-1H-pyrazole-5- | | | 7.69(dd, J=8.69, 1.85Hz, 1H) |
| | carboxamide | | | 7.93(d, J=8.79Hz, 1H) |
| | | | | 8.43(d, J=1.95Hz, 1H) |
| 8 | 4-(Ethoxymethyl)-N-[2- | 343.1 | 343.0 | (400MHz, methanol-D₄) δppm |
| | (hydroxymethyl)-1,3- | | | 1.24(t, J=7.03Hz, 3H) |
| | benzothiazol-5- | | | 3.58(q, J=7.03Hz, 2H) |
| | yl]benzamide | | | 4.58(s, 2H) 4.95(s, 2H) 7.48(d, |
| | | | | J=8.59Hz, 2H) 7.71(dd, |
| | | | | J=8.69, 2.05Hz, 1H) 7.92(s, |
| | | | | 1H) 7.94(d, J=7.81Hz, 2H) |
| | | | | 8.41(d, J=1.95Hz, 1H) |
| 9 | N-[2-(Hydroxymethyl)- | 335.1 | 335.0 | (400MHz, chloroform-D) δppm |
| | 1,3-benzothiazol-5-yl]-1- | | | 2.78(s, 3H) 6.79(d, |
| | phenyl-1H-pyrazole-5- | | | J=1.76Hz, 1H) 7.35(m, 5H) |
| | carboxamide | | | 7.60(m, 3H) 7.89(s, 1H) |
| 10 | 4-Bromo-N-[2- | 377.0 | 377.0 | (400MHz, methanol-D₄) δppm |
| | (hydroxymethyl)-1,3- | | | 2.45(s, 3H) 4.95(s, 2H) |
| | benzothiazol-5-yl]-2- | | | 7.40(d, J=8.20Hz, 1H) |
| | methylbenzamide | | | 7.45(m, 1H) 7.49(s, 1H) |
| | | | | 7.66(dd, J=8.69, 2.05Hz, 1H) |
| | | | | 7.93(d, J=8.59Hz, 1H) |
| | | | | 8.40(d, J=1.95Hz, 1H) |
| 11 | 4-tert-Butoxy-N-(2- | 325.1 | 325.2 | (400MHz, chloroform-D) [symbol] ppm |
| | methyl-1,3-benzoxazol-5- | | | 1.41(s, 9H) 2.63(s, 3H) |
| | yl) benzamide | | | 7.07(m, 2H) 7.43(d, |
| | | | | J=8.79Hz, 1H) 7.58(dt, |
| | | | | J=8.79, 2.15Hz, 1H) 7.82(m, |
| | | | | 2H) 7.89(d, J=1.95Hz, 1H) |
| | | | | 7.98(s, 1H) |
| 12 | N-(4-Bromo-2-methyl-1,3- | 403.0 | 403.0 | (400MHz, chloroform-D) δppm |
| | benzothiazol-5-yl)-4-tert- | | | 1.38(s, 9H) 2.90(s, 3H) |
| | butylbenzamide | | | 7.56(d, J=8.59Hz, 2H) |
| | | | | 7.79(d, J=8.79Hz, 1H) 7.93(d, |
| | | | | J=8.59Hz, 2H) 8.63(d, |
| | | | | J=8.98Hz, 1H) |
| 13 | 4-tert-Butyl-N-(4,7- | 481.0 | 480.7 | (400MHz, methanol-D₄) δppm |
| | dibromo-2-methyl-1,3- | | | 1.37(s, 9H) 2.85(s, 3H) |
| | benzothiazol-5- | | | 7.58(d, J=8.40Hz, 2H) |
| | yl)benzamide | | | 7.97(d, J=8.40Hz, 2H) 8.31(s, 1H) |
| 14 | N-[2-(hydroxymethyl)-1,3- | 419.1 | 419.0 | (400MHz, methanol-D4) δppm |
| | benzothiazol-5-yl]-1- | | | 4.95(m, 2H), 7.59(m, 6H), |
| | phenyl-5- | | | 7.96(m, 1H), 8.14(m, 1H), |
| | (trifluoromethyl)-1H- | | | 8.37(m, 1H). |
| | pyrazole-4-carboxamide | | | |
| 15 | 4-Iodo-N-(2-methyl-5- | 395.0 | 394.8 | (400MHz, chloroform-D) δppm |
| | benzothiazolyl)benzamide | | | 2.84(s, 3H) 7.64(d, |
| | | | | J=8.59Hz, 2H) 7.73(dd, |
| | | | | J=8.59, 1.95Hz, 1H) 7.80(m, |
| | | | | 1H) 7.86(d, J=8.59Hz, 2H) |
| | | | | 7.94(m, 1H) 8.14(d, J=1.95Hz, |
| | | | | 1H) |
| 16 | 4-(tert-Butoxymethyl)-N- | 371.1 | 371.0 | (400MHz, methanol-D₄) δppm |
| | [2-(hydroxymethyl)-1,3- | | | 1.30(s, 9H) 4.54(s, 2H) |
| | benzothiazol-5- | | | 4.94(s, 2H) 7.48(d, J=8.59Hz, |
| | yl]benzamide | | | 2H) 7.71(dd, J=8.69, |
| | | | | 2.05Hz, 1H) 7.92(m, 3H) |
| | | | | 8.39(d, J=1.56Hz, 1H) |
| 17 | N-(1,2-Dimethyl-1H- | 392.0 | 392.0 | (400MHz, DMSO-D₆) δppm |
| | benzimidazol-5-yl)-4- | | | 2.75(s, 3H) 3.87(s, 3H) |
| | iodobenzamide | | | 7.75(d, J=8.59Hz, 2H) 7.80(t, |
| | | | | J=2.05Hz, 1H) 7.84(d, |
| | | | | J=8.98Hz, 1H) 7.92(d, |
| | | | | J=8.40Hz, 2H) 8.32(t, |
| | | | | J=2.15Hz, 1H) 10.58(s, 1H) |
| 18 | N-[2-(Hydroxymethyl)- | 450.1 | 451.0 | (400MHz, methanol-D4) δppm |
| | 1,3-benzothiazol-5-yl]-4- | | | 4.95(s, 2H) 7.72(d, |
| | [2,2,2-trifluoro-1-hydroxy- | | | J=8.59Hz, 1H) 7.90(d, |
| | 1- | | | J=8.40Hz, 2H) 7.96(d, |
| | (trifluoromethyl)ethyl]benzamide | | | J=8.59Hz, 1H) 8.04(d, |
| | | | | J=8.59Hz, 2H) |
| | | | | 8.40-8.46(m, 1H) 10.43(s, 1H) |
| 19 | N-[2-(Hydroxymethyl)- | 343.1 | 343.0 | (400MHz, methanol-D4) δppm |
| | 1,3-benzothiazol-5-yl]-4- | | | 1.31(d, J=6.05Hz, 6H) |
| | isopropoxybenzamide | | | 4.65(dt, J=11.96, 6.03Hz, 1H) |
| | | | | 4.93(s, 2H) 6.95(d, |
| | | | | J=8.98Hz, 2H) 7.66(dd, |
| | | | | J=8.59, 1.76Hz, 1H) |
| | | | | 7.85-7.91(m, 2H) 8.36(s, 1H) |
| 20 | 4-Bromo-2-chloro-N-[2- | 397.0 | 396.7 | (400MHz, DMSO-D6) δppm |
| | (hydroxymethyl)-1,3- | | | 4.82(s, 2H) 6.23(t, J=12.11, |
| | benzothiazol-5- | | | 6.05Hz, 1H) 7.58(d, J=8.20Hz, |
| | yl]benzamide | | | 1H) 7.63(dt, J=8.79, |
| | | | | 3.32, 2.15Hz, 1H) 7.68(dd, |
| | | | | J=8.20, 1.95Hz, 1H) 7.88(d, |
| | | | | J=1.95Hz, 1H) 8.01(d, |
| | | | | J=8.59Hz, 1H) 8.35(t, |
| | | | | J=1.66Hz, 1H) 10.70(s, 1H) |
| 21 | 4-Bromo-2-fluoro-N-[2- | 381.0 | 381.0 | (400MHz, chloroform-D) δppm |
| | (hydroxymethyl)-1,3- | | | 5.09(s, 2H) 7.43(d, |
| | benzothiazol-5- | | | J=11.52Hz, 1H) 7.50(dd, |
| | yl]benzamide | | | J=8.40, 1.56Hz, 1H) 7.71(s, |
| | | | | 1H) 7.88(d, J=8.59Hz, 1H) |
| | | | | 8.11(t, J=8.49Hz, 1H) |
| | | | | 8.33(s, 1H) 8.52(s, 1H) |
| 22 | N-[2-(Hydroxymethyl)- | 384.1 | 384.0 | (400MHz, methanol-D4) δppm |
| | 1,3-benzothiazol-5-yl]-4- | | | 3.18-3.46(m, 4H) |
| | (morpholin-4- | | | 3.75(s, 2H) 3.94-4.17(m, 2H) |
| | ylmethyl)benzamide | | | 4.47(s, 2H) 4.96(s, 2H) |
| | | | | 7.65-7.75(m, 3H) 7.98(d, J=8.79Hz, |
| | | | | 1H) 8.10(d, J=8.20Hz, 2H) |
| | | | | 8.42(d, J=1.56Hz, 1H) |
| 23 | 3-Fluoro-N-[2- | 371.0 | 371.0 | (400MHz, methanoL-D4) δppm |
| | (hydroxymethyl)-1,3- | | | 4.95(s, 2H) 7.73(dd, |
| | benzothiazol-5-yl]-4- | | | J=8.69, 1.86Hz, 1H) |
| | (trifluoromethyl)benzamide | | | 7.83-8.01(m, 4H) 8.43(d, J=1.76Hz, |
| | | | | 1H) |
| 24 | 4-tert-butoxy-N-[4-chloro- | 391.1 | 391.0 | (400MHz, methanol-D4) δppm |
| | 2-(hydroxymethyl)-1,3- | | | 1.42(s, 9H) 4.98(s, 2H) |
| | benzothiazol-5- | | | 7.13(d, J=8.59Hz, 2H) |
| | yl]benzamide | | | 7.75(d, J=8.59Hz, 1H) |
| | | | | 7.88-7.98(m, 3H) |
| 25 | 4-(tert-Butoxymethyl)-N- | 405.1 | 405.0 | (400MHz, methanol-D4) δppm |
| | [4-chloro-2- | | | 1.26-1.36(m, 9H) |
| | (hydroxymethyl)-1,3- | | | 4.57(s, 2H) 4.98(s, 2H) 5.48(s, 1H) |
| | benzothiazol-5- | | | 7.51(d, J=8.20Hz, 2H) |
| | yl]benzamide | | | 7.75(d, J=8.59Hz, 1H) |
| | | | | 7.97(dd, J=8.40, 2.73Hz, 3H) |
| 26 | 3-Fluoro-N-(2-methyl-1,3- | 354.3 | 355.0 | |
| | benzothiazol-5-yl)-4- | | | |
| | trifluoromethyl-benzamide | | | |
| 27 | 2-tert-Butyl-5-methyl-2H- | 328.4 | 329 | |
| | pyrazole-3-carboxylic acid | | | |
| | (2-methyl-1,3- | | | |
| | benzothiazol-5-yl)-amide | | | |
| 28 | 2-Fluoro-N-(2-methyl-1,3- | 354.3 | 355.0 | |
| | benzothiazol-5-yl)-4- | | | |
| | trifluoromethyl-benzamide | | | |
| 29 | 2-Fluoro-N-(2-methyl-1,3- | 354.3 | 354.0 | |
| | benzothiazol-5-yl)-3- | | | |
| | trifluoromethyl-benzamide | | | |
| 30 | 4-Fluoro-N-(2-methyl-1,3- | 354.3 | 354.0 | |
| | benzothiazol-5-yl)-3- | | | |
| | trifluoromethyl-benzamide | | | |
| 31 | 3,4-Dimethyl-N-(2- | 296.4 | 297.1 | |
| | methyl-benzothiazol-5-yl)- | | | |
| | benzamide | | | |
| 32 | 2,2-Difluoro- | 348.3 | 349 | |
| | benzo[1,3]dioxole-5- | | | |
| | carboxylic acid (2-methyl- | | | |
| | 1,3-benzothiazol-5-yl)- | | | |
| | amide | | | |
| 33 | N-(2-Methyl-1,3- | 337.3 | 338 | |
| | benzothiazol-5-yl)-6- | | | |
| | trifluoromethyl- | | | |
| | nicotinamide | | | |
| 34 | N-(2-Methyl-1,3- | 310.4 | 311.1 | |
| | benzothiazol-5-yl)-4- | | | |
| | propyl-benzamide | | | |
| 35 | 3-Iodo-N-(2-methyl-1,3- | 394.23 | 394.9 | |
| | benzothiazol-5-yl)- | | | |
| | benzamide | | | |
| 36 | 2,5-Dimethyl-furan-3- | 286.35 | 287 | |
| | carboxylic acid (2-methyl- | | | |
| | 1,3-benzothiazol-5-yl)- | | | |
| | amide | | | |
| 37 | 5-tert-Butyl-2-methyl- | 328.43 | 329.1 | |
| | furan-3-carboxylic acid (2- | | | |
| | methyl-1,3-benzothiazol- | | | |
| | 5-yl)-amide | | | |
| 38 | 4-Bromo-3-methyl-N-(2- | 361.26 | 360.99 | |
| | methyl-1,3-benzothiazol- | | | |
| | 5-yl)-benzamide | | | |
| 39 | 3,4-Difluoro-N-(2-methyl- | 304.32 | 305 | |
| | 1,3-benzothiazol-5-yl)- | | | |
| | benzamide | | | |
| 40 | 3-Chloro-2-fluoro-N-(2- | 320.77 | 321 | |
| | methyl-1,3-benzothiazol- | | | |
| | 5-yl)-benzamide | | | |
| 41 | Pyridine-2-carboxylic acid | 269.33 | 270 | |
| | (2-methyl-1,3- | | | |
| | benzothiazol-5-yl)-amide | | | |
| 42 | 2-Benzyl-5-tert-butyl-2H- | 404.54 | 405.1 | |
| | pyrazole-3-carboxylic acid | | | |
| | (2-methyl-1,3- | | | |
| | benzothiazol-5-yl)-amide | | | |
| 43 | 3-Fluoro-4- | 391.25 | 392 | |
| | trifluoromethyl-N-(2- | | | |
| | trifluoromethyl-1H- | | | |
| | benzimidazol-5-yl)- | | | |
| | benzamide | | | |
| 44 | 2-Fluoro-5- | 391.25 | 392 | |
| | trifluoromethyl-N-(2- | | | |
| | trifluoromethyl-1H- | | | |
| | benzimidazol-5-yl)- | | | |
| | benzamide | | | |
| 45 | 4-Chloro-N-(2-methyl- | 302.8 | 302.9 | (400MHz, DMSO-D6) δppm |
| | benzothiazol-5-yl)- | | | 2.8(s, 3H) 7.6(d, J=8.6Hz, 2H) |
| | benzamide | | | 7.8(d, J=9.1Hz, 1H) |
| | | | | 8.0(m, 3H) 8.4(s, 1H) 10.5(s, 1H) |
| 46 | 1-Phenyl-5- | 402.4 | 402.9 | (400MHz, DMSO-D6) δppm |
| | trifluoromethyl-1H- | | | 2.8(s, 3H) 7.5(m, 2H) |
| | pyrazole-3-carboxylic acid | | | 7.6(m, 3H) 7.7(d, J=9.1Hz, 1H) |
| | (2-methyl-1,3- | | | 8.0(d, J=8.6Hz, 1H) |
| | benzothiazol-5-yl)-amide | | | 8.3(m, 2H) 10.7(s, 1H) |
| 47 | 1-Phenyl-5-propyl-1H- | 376.5 | 376.9 | (400MHz, chloroform-D) δppm |
| | pyrazole-4-carboxylic acid | | | 0.8(t, J=7.3Hz, 3H) |
| | (2-methyl-1,3- | | | 1.5(m, 2H) 2.8(s, 3H) 2.9(m, 2H) |
| | benzothiazol-5-yl)-amide | | | 7.4(m, 2H) 7.4(m, 3H) |
| | | | | 7.7(s, 1H) 7.7(m, 2H) |
| | | | | 7.9(s, 1H) 8.0(m, 1H) |
| 48 | 2,3-Difluoro-N-(2-methyl- | 372.3 | 372.7 | (400MHz, chloroform-D) δppm |
| | 1,3-benzothiazol-5-yl)-4- | | | 2.8(s, 3H) 7.5(t, J=7.3Hz, |
| | trifluoromethyl-benzamide | | | 1H) 7.7(dd, J=8.6, 2.0Hz, |
| | | | | 1H) 7.8(d, J=8.6Hz, 1H) |
| | | | | 8.0(t, J=7.6Hz, 1H) |
| | | | | 8.2(d, J=2.0Hz, 1H) 8.4(d, |
| | | | | broad, 1H) |
| 49 | 3-Fluoro-4-methyl-N-(2- | 300.4 | 300.8 | |
| | methyl-1,3-benzothiazol- | | | |
| | 5-yl)-benzamide | | | |
| 50 | 4-tert-Butyl-N-(2-methyl- | 324.5 | 325.2 | (400MHz, chloroform-D) δppm |
| | 1,3-benzothiazol-5-yl)- | | | 1.4(s, 9H) 2.8(s, 3H) |
| | benzamide | | | 7.5(d, J=8.6Hz, 2H) 7.8(m, |
| | | | | 2H) 7.8(d, J=8.6Hz, 2H) |
| | | | | 7.9(s, 1H) 8.1(m, 1H) |
| 51 | 4-Ethyl-N-(2-methyl-1,3- | 296.4 | 297.2 | |
| | benzothiazol-5-yl)- | | | |
| | benzamide | | | |
| 52 | 4-tert-Butyl-N-(2-methyl- | 308.4 | 309 | |
| | 1,3-benzooxazol-5-yl)- | | | |
| | benzamide | | | |
| 53 | Biphenyl-4-carboxylic | 344.4 | 345 | |
| | acid (2-methyl-1,3- | | | |
| | benzothiazol-5-yl)-amide | | | |
| 54 | 3-Bromo-thiophene-2- | 353.3 | 354 | (400MHz, chloroform-D) δppm |
| | carboxylic acid (2-methyl- | | | 2.84(s, 3H) 7.11(d, |
| | 1,3-benzothiazol-5-yl)- | | | J=5.27Hz, 1H) 7.54(d, |
| | amide | | | J=5.27Hz, 1H) 7.73(dd, |
| | | | | J=8.79, 2.15Hz, 1H) 7.80(d, |
| | | | | J=8.59Hz, 1H) 8.20(d, |
| | | | | J=1.95Hz, 1H) 8.98(s, 1H) |
| 55 | 4-Bromo-2-methyl-N-(2- | 361.3 | 362 | (400MHz, chloroform-D) δppm |
| | methyl-1,3-benzothiazol- | | | 2.50(s, 3H) 2.84(s, 3H) |
| | 5-yl)-benzamide | | | 7.42(d, J=19.14Hz, 2H) |
| | | | | 7.64(s, 1H) 7.77(m, 3H) 8.10(s, |
| | | | | 1H) |
| 56 | 4-tert-Butoxy-N-(2- | 340.4 | 341 | (400MHz, chloroform-D) δppm |
| | methyl-1,3-benzothiazol- | | | 1.41(m, 9H), 2.84(s, 3H), |
| | 5-yl)-benzamide | | | 7.09(d, J=8.79Hz, 2H), |
| | | | | 7.78(m, 2H), 7.83(d, J=8.98Hz, |
| | | | | 2H), 7.93(s, 1H), |
| | | | | 8.12(m, 1H) |
| 57 | 2-Chloro-3,4-dimethoxy- | 362.8 | 363 | (400MHz, chloroform-D) δppm |
| | N-(2-methyl-1,3- | | | 2.84(s, 3H), 3.90(s, 3H), |
| | benzothiazol-5-yl)- | | | 3.94(s, 3H), 6.94(d, |
| | benzamide | | | J=8.79Hz, 1H), 7.62(d, |
| | | | | J=8.79Hz, 1H), 7.78(m, 2H), |
| | | | | 8.18(s, 1H). |
| 58 | 4-Iodo-N-(2-methyl-1,3- | 394.2 | 395 | (400MHz, chloroform-D) δppm |
| | benzothiazol-5-yl)- | | | 2.84(s, 3H) 7.64(d, |
| | benzamide | | | J=8.59Hz, 2H) 7.73(dd, |
| | | | | J=8.59, 1.95Hz, 1H) 7.80(m, |
| | | | | 1H) 7.86(d, J=8.59Hz, 2H) |
| | | | | 7.94(m, 1H) 8.14(d, J=1.95Hz, |
| | | | | 1H) |
| 59 | 4-Amino-N-(2-methyl-1,3- | 328.4 | 329 | (400MHz, DMSO-D6) δppm |
| | benzothiazol-5-yl)-3-nitro- | | | 2.79(s, 3H), 7.10(d, J=8.98Hz, |
| | benzamide | | | 1H), 7.75(m, 1H), |
| | | | | 7.87(s, 2H), 7.96(d, J=8.59Hz, 1H), |
| | | | | 8.01(dd, J=8.89, 2.25Hz, |
| | | | | 1H), 8.39(m, 1H), 8.75(d, |
| | | | | J=2.15Hz, 1H), 10.37(s, 1H) |
| 60 | N-(2-Methyl-1,3- | 294.4 | 295 | (400MHz, chloroform-D) δppm |
| | benzothiazol-5-yl)-4- | | | 2.84(s, 3H) 5.40(d, |
| | vinyl-benzamide | | | J=10.94Hz, 1H) 5.87(d, |
| | | | | J=17.57Hz, 1H) 6.78(dd, |
| | | | | J=17.57, 10.94Hz, 1H) |
| | | | | 7.52(d, J=8.20Hz, 2H) 7.78(m, 2H) |
| | | | | 7.87(d, J=8.40Hz, 2H) |
| | | | | 7.99(s, 1H) 8.15(s, 1H) |
| 61 | 4-Ethoxy-N-(2-methyl- | 312.4 | 313 | (400MHz, chloroform-D) δppm |
| | 1,3-benzothiazol-5-yl)- | | | 1.45(t, J=7.03Hz, 3H) |
| | benzamide | | | 2.83(s, 3H) 4.10(q, J=14.06, |
| | | | | 7.03Hz, 2H) 6.96(d, J=8.98Hz, |
| | | | | 2H) 7.77(m, 2H) |
| | | | | 7.88(d, J=6.83Hz, 2H) 7.98(s, 1H) |
| | | | | 8.11(m, 1H) |
| 62 | 4-Ethylsulfanyl-N-(2- | 328.5 | 329 | (400MHz, chloroform-D) δppm |
| | methyl-1,3-benzothiazol- | | | 1.37(t, J=7.42Hz, 3H) |
| | 5-yl)-benzamide | | | 2.83(s, 3H) 3.02(q, J=14.65, |
| | | | | 7.22Hz, 2H) 7.33(d, J=8.79Hz, |
| | | | | 2H) 7.76(s, 2H) 7.81(d, |
| | | | | J=8.59Hz, 2H) 8.13(s, 2H) |
| 63 | 4-Dimethylamino- | 361.5 | 362 | (400MHz, chloroform-D) δppm |
| | naphthalene-1-carboxylic | | | 2.85(s, 3H), 2.96(m, 6H), |
| | acid (2-methyl-1,3- | | | 7.03(d, J=7.81Hz, 1H), |
| | benzothiazol-5-yl)-amide | | | 7.54(m, 2H), 7.72(d, J=7.81Hz, |
| | | | | 1H), 7.83(m, 3H), |
| | | | | 8.13(m, 1H), 8.26(m, 1H), |
| | | | | 8.43(m, 1H). |
| 64 | 2-Fluoro-6-iodo-N-(2- | 412.2 | 413 | |
| | methyl-1,3-benzothiazol- | | | |
| | 5-yl)-benzamide | | | |
| 65 | 4-Ethoxymethyl-N-(2- | 326.4 | 327 | (400MHz, chloroform-D) δppm |
| | methyl-1,3-benzothiazol- | | | 1.27(t, J=14.06, 7.03Hz, |
| | 5-yl)-benzamide | | | 3H) 2.84(s, 3H) 3.58(q, |
| | | | | J=14.06, 7.03Hz, 2H) |
| | | | | 4.58(s, 2H) 7.47(d, J=8.59Hz, 2H) |
| | | | | 7.78(m, 2H) 7.88(d, |
| | | | | J=8.40Hz, 2H) 8.05(s, 1H) |
| | | | | 8.14(s, 1H) |
| 66 | N-(2-Methyl-1,3- | 352.3 | 353 | (400MHz, chloroform-D) δppm |
| | benzothiazol-5-yl)-4- | | | 2.84(s, 3H) 7.33(d, |
| | trifluoromethoxy- | | | J=8.79Hz, 2H) 7.77(m, 2H) |
| | benzamide | | | 7.95(d, J=8.98Hz, 2H) |
| | | | | 8.00(s, 1H) 8.14(d, J=1.95Hz, 1H) |
| 67 | 4-Chloro-3-fluoro-N-(2- | 320.8 | 321 | (400MHz, DMSO-D6) δppm |
| | methyl-1,3-benzothiazol- | | | 2.74(s, 3H), 7.70(m, 1H), |
| | 5-yl)-benzamide | | | 7.75(m, 1H), 7.82(dd, |
| | | | | J=8.40, 1.95Hz, 1H), |
| | | | | 7.96(m, 2H), 8.396(m, 1H), |
| | | | | 10.49(s, 1H) |
| 68 | 4-tert-Butyl-N-(2-formyl- | 338.4 | 339 | (400MHz, DMSO-D6) [symbol] |
| | 1,3-benzothiazol-5-yl)- | | | 1.35(s, 9H), 7.27(d, J=8.6Hz, 2H), |
| | benzamide | | | 7.60-7.73(m, 2H), 7.73(d, |
| | | | | J=8.6Hz, 1H), 7.86(s, 1H), |
| | | | | 8.13(s, 1H), 8.84(s, 1H) |
| 69 | 4-tert-Butyl-N-(2- | 340.5 | 341 | (400MHz, DMSO-D6) δ |
| | hydroxymethyl-1,3- | | | 1.30(s, 9H), 3.12(s, 1H), 4.42(s, |
| | benzothiazol-5-yl)- | | | 2H) 7.22(d, J=8.6Hz, 2H), |
| | benzamide | | | 7.62-7.76(m, 2H), 7.83(d, |
| | | | | J=8.6Hz, 1H), 7.96(s, 1H), |
| | | | | 8.25(s, 1H) |

### Example 70

### 4-tert-Butyl-N-(2-{[(2-methoxypyridin-3-yl)amino]methyl}-1,3-benzothiazol-5-yl)benzamide

A mixture of SeO₂ (4.44 g, 40.0 mmol) and 4-tert-butyl-N-(2-methyl-benzothiazol-5-yl)-benzamide (16.0 mmol) in dioxane (20.0 mL) was kept under a N₂ atmosphere and heated to 100° C. for 18 hours with vigorous stirring. After cooling to room temperature, the dioxane was removed by evaporation under reduced pressure. The resulting residue was dissolved in EtOAc, washed with brine, dried with Na₂SO₄, filtered and concentrated under reduced pressure to yield the aldehyde, MS (ESI⁺) m/z 325.0 [M+H]⁺. The aldehyde (100 mg, 0.300 mmol) was mixed with 2-methoxypyridin-3-amine (36.0 mg, 0.300 mmol) and MgSO₄ (100 mg) in THF (3.00 mL). After 18 hours, B₁₀H₄ (14.0 mg, 0.320 mmol) dissolved in MeOH (3.00 mL) was added. The mixture was stirred until the reaction appeared complete by TLC analysis. 1M NaOH was added and the solvents were evaporated. The residue was purified by flash chromatography eluting with mixtures of hexanes and EtOAc (4:1, 1:1). ¹H NMR (400 MHz, chloroform-D) δ ppm 1.29 (s, 9 H) 3.98 (s, 3 H) 4.68 (d, J=5.86 Hz, 2 H) 6.67 (m, 2 H) 7.40 (dt, J=8.69, 2.10 Hz, 2 H) 7.51 (dd, J=4.69, 1.95 Hz, 1 H) 7.66 (d, J=1.17 Hz, 2 H) 7.80 (ddd, J=8.59, 2.25, 2.05 Hz, 2 H) 8.25 (d, J=1.17 Hz, 1 H) 8.45 (s, 1 H); MS [M+H] calc. 447.2 found 447.0.

### Example 71

### 4-tert-Butyl-N-[2-(1-hydroxyethyl)-1,3-benzothiazol-5-yl]benzamide

Methylmagnesium bromide (276 uL, 3.0 M in Et₂O) was added dropwise via syringe to a stirred solution of the aldehyde (obtained as an intermediate in Example 70) (100 mg, 0.30 mmol) in THF (10.0 mL) at −78° C. under nitrogen. After addition was complete the mixture was stirred for additional 1 hour and quenched with saturated aqueous ammonium chloride (2.0 mL). The mixture was diluted with EtOAc (25.0 mL) and water (20.0 mL) and the organic phase was seaprated. The aqueous phase was extracted with EtOAc (2×10.0 mL) and the organic phases combined and washed with brine solution (30.0 mL). The organic was dried with Na₂SO₄, filtered and concentrated by rotary evaporator to a residue which was purified by column chromatography on silica gel using EtOAc/hexanes as an eluent to yield the title product. ¹H NMR (400 MHz, methanol-D4) δ ppm 1.34 (s, 9 H), 1.62 (d, J=6.44 Hz, 3 H), 5.12 (m, 1 H), 7.54 (d, J=8.59 Hz, 2 H), 7.69 (dd, J=8.69, 2.05 Hz, 1 H), 7.89 (m, 3 H), 8.39 (d, J=1.95 Hz, 1 H). MS [M+H] calc. 355.1 found 355.2.

### Example 72

### 4-tert-Butyl-N-{2-[(1H-pyrazol-3-ylamino)methyl]-1,3-benzothiazol-5-yl}benzamide

The title compounds wa synthesized according to the procedure described in Example 70 using 1H-pyrazol-3-amine at the reductive amination step. ¹H NMR (400 MHz, methanol-D4) δ 1.31 (m, 9 H) 4.71 (s, 2 H) 5.62 (d, J=2.34 Hz, 1 H) 7.35 (d, J=2.34 Hz, 1 H) 7.53 (d, J=8.79 Hz, 2 H) 7.67 (dd, J=8.69, 2.05 Hz, 1 H) 7.83 (d, J=8.59 Hz,1 H) 7.88 (d, J=8.79 Hz, 2 H) 8.37 (d, J=1.76 Hz, 1 H); MS [M+H] calc. 406.2 found 406.0.

### Example 73

### 4-(1,1-Dimethylethyl)-N-[2-[[(4-nitrophenyl)amino]methyl]-5-benzothiazolyl]-benzamide

The title compound was synthesized according to the procedure described in Example 70 using p-nitroaniline at the reductive amination step. ¹H NMR (400 MHz, DMSO-D6) δ ppm 1.32 (s, 9 H), 4.91 (m, 2 H), 6.78 (d, J=9.18 Hz, 2 H), 7.56 (d, J=8.40 Hz, 2 H), 7.76 (dd, J=8.79, 1.95 Hz, 1 H), 7.91 (d, J=8.40 Hz, 2 H), 7.97 (d, J=8.79 Hz, 1 H), 8.02 (d, J=9.18 Hz, 2 H), 8.17 (t, J=6.25 Hz, 1 H), 8.50 (d, J=1.76 Hz, 1 H), 10.38 (s, 1 H). MS [M+H] calc. 461.2 found 461.0.

### Example 74

### N-[2-(Aminomethyl)-1,3-benzothiazol-5-yl]-4-tert-butylbenzamide

4-tert-Butyl-N-(2-hydroxymethyl-1,3-benzothiazol-5-yl)-benzamide (44.0 mg, 0.380 mmol) was mixed with MsCl (40.0 mg, 0.390 mmol, 0.0540 mL) and Et₃N (51.0 mg, 0.500 mmol) in DCM (5.00 mL) and the solution was stirred for 10 minutes. NH₃ (2.0M in EtOH) was added, and the mixture was stirred for additional 18 hours. The solvent was evaporated, and the crude product was purified by HPLC (Luna 15 u, C18 (2), 250 mm×21.2 mm) eluting with mixtures of MeCN and H₂O containing 1% TFA. ¹H NMR (400 MHz, methanol-D4) δ ppm 1.21 (s, 9 H) 4.48 (s, 2 H) 7.41 (d, J=8.20 Hz, 2 H) 7.57 (d, J=8.20 Hz, 1 H) 7.76 (d, J=8.20 Hz, 2 H) 7.83 (d, J=8.59 Hz, 1 H) 8.47 (s, 1 H); MS [M+H] calc. 340.1 found 340.3.

### Example 75

### 4-tert-Butyl-N-(2-{[(methylsulfonyl)amino]methyl}-1,3-benzothiazol-5-yl)benzamide

N-[2-(Aminomethyl)-1,3-benzothiazol-5-yl]-4-tert-butylbenzamide (example 74) (130 mg, 0.384 mmol) was stirred with MsCl (44.0 mg, 0.387 mmol) and Et₃N (58.0 mg, 0.600 mmol, 0.0800 mL) in DCM (5.00 mL) for 1 hour. The solvent was evaporated, and the residue was purified by HPLC (Luna 15 u, C18 (2), 250 mm×21.2 mm) eluting with mixtures of MeCN and H₂O containing 1% TFA to yield the title product. ¹H NMR (400 MHz, chloroform-D) δ ppm 1.35 (s, 9 H) 3.05 (s, 3 H) 4.79 (s, 2 H) 5.73 (s, 1 H) 7.52 (d, J=8.59 Hz, 2 H) 7.80 (s, 2 H) 7.85 (d, J=8.40 Hz, 2 H) 8.12 (s, 1 H) 8.23 (s, 1 H); MS [M+] calc. 417.5 found 417.9; Anal. found C 54.39% H 5.43% N 8.71%.

### Example 76

### N-{2-[(Acetylamino)methyl]-1,3-benzothiazol-5-yl}-4-tert-butylbenzamide

N-[2-(Aminomethyl)-1,3-benzothiazol-5-yl]-4-tert-butylbenzamide (example 74) (60.0 mg, 0.18 mmol) was stirred with acetyl chloride (16.0 mg, 0.2 mmol, 0.015 mL) and Et₃N (25.0 mg, 0.25 mmol) in DCM (5.00 mL) for 1 hour. The solvent was evaporated, and the residue was purified by HPLC eluting with mixtures of MeCN and H₂O containing 1% TFA to yield the title product. ¹H NMR (400 MHz, chloroform-D) δ ppm 1.35 (s, 9 H) 2.12 (s, 3 H) 4.84 (s, 2 H) 7.49 (d, J=8.40 Hz, 2 H) 7.68 (s, 1 H) 7.75 (d, J=8.79 Hz, 1 H) 7.84 (d, J=8.20 Hz, 2 H) 8.20 (s, 1 H) 8.64 (s, 1 H) 11.35 (s, 1 H); MS [M+H] calc. 382.1 found 382.0; Anal. found C 55.85% H 4.94% N 8.60%.

### Example 77

### 5-[(4-tert-Butylbenzoyl)amino]-1,3-benzothiazole-2-carboxamide

The aldehyde (example 70) (100 mg, 0.3 mmol) was dissolved in THF (10.0 mL) and a mixture of sodium chlorite (54.0 mg, 0.6 mmol) and sulfamic acid (58.0 mg, 0.6 mmol) in H₂O (5.0 mL) was added drop-wise. The mixture was stirred for 1 hour, and then the aqueous phase was extracted with EtOAc. The combined organic phases were dried over MgSO₄, filtered and evaporated to yield the acid, which was immediately dissolved in DCM (5.0 mL) containing a mixture of allyl chloroformate (48.0 mg, 0.400 mmol) and DMAP (48.0 mg, 0.400 mmol, 0.340 mL). The mixture was stirred for 1 hour and then evaporated to yield the mixed anhydride: MS [M+] calc. 435.0 found 435.9. The anhydride was dissolved in 5.0 mL of EtOH containing NH₃ (2.0M), and the mixture was stirred for 18 hours. The solvent was evaporated, and the product was purified by flash chromatography eluting with mixtures of hexanes and EtOAc (4:1, 1:1) to yield decarboxylated material (see example 77) and the title product. ¹H NMR (400 MHz, chloroform-D) δ ppm 1.34 (m, 9 H) 6.11 (s, 2 H) 7.41 (s, 1 H) 7.49 (d, J=7.62 Hz, 2 H) 7.73 (d, J=8.59 Hz, 1 H) 7.86 (d, J=7.23 Hz, 2 H) 8.33 (m, 1 H) 8.49 (s, 1 H); MS [M+H] calc. 354.1 found 354.0.

### Example 78

### N-1,3-Benzothiazol-5-yl-4-tert-butylbenzamide

See above (example 76). ¹H NMR (400 MHz, DMSO-D₆) δ ppm 1.30 (s, 9 H) 7.54 (d, J=8.40 Hz, 2 H) 7.82 (dd, J=8.79, 1.95 Hz, 1 H) 7.90 (d, J=8.59 Hz, 2 H) 8.08 (d, J=8.79 Hz, 1 H) 8.59 (d, J=1.95 Hz, 1 H) 9.36 (s, 1H) 10.38 (s, 1 H); 1R (neat) 1661 cm⁻¹; MS [M+H] calc. 311.1 found 311.0.

### Example 79

### 4-Chloro-N-[2-(hydroxymethyl)-1,3-benzothiazol-5-yl]benzamide

According to amide bond forming procedure described in Example 2, 5-amino-2-methylbenzothiazole reacted with 4-chlorobenzoyl chloride to yield 4-chloro-N-(2-methyl-benzothiazol-5-yl)-benzamide: MS [M+] calc. 302, found 302.0. This intermediate was oxidized with SeO₂ to the corresponding aldehyde as described in Example 70. The aldehyde (3.30 mmol) was mixed with NaBH₄ (122 mg, 3.30 mmol) in MeOH (150 mL). After the reaction was complete according to TLC, the volatiles were removed and the residue was dissolved in a mixture of DCM and MeOH (10 mL, 1:5) and passed through a short pad of silica. The filtrate was concentrated and a residue was crystallized from a mixture of EtOAc and MeOH (40:1). A yellow solid formed was collected by filtration. ¹H NMR (400 MHz, DMSO-D6) δ ppm 4.85 (m, 2 H), 6.26 (t, J=5.96 Hz, 1 H), 7.62 (d, J=8.40 Hz, 2 H), 7.76 (m, 1 H), 8.02 (m, 3 H), 8.43 (m, 1 H), 10.50 (s, 1 H). MS [M+H] calc. 319.0 found 319.0.

### Example 80

### 1-(4-chlorophenyl)-N-[2-(hydroxymethyl)-1,3-benzothiazol-5-yl]-5-propyl-1H-pyrazole-4-carboxamide

The title compound was synthesized from 5-amino-2-methylbenzothiazole and 1-(4-chlorophenyl)-5-propyl-1H-pyrazole-4-carbonyl chloride according to the procedure described in the example 79. ¹H NMR (400 MHz, DMSO-D6) δ ppm 0.76 (t, J=7.32 Hz, 3 H), 1.46 (m, 2 H), 2.97 (m, 2 H), 4.85 (d, J=6.05 Hz, 2 H), 6.26 (t, J=5.96 Hz, 1 H), 7.55 (d, J=8.79 Hz, 2 1), 7.65 (d, J=8.79 Hz, 2 H), 7.74 (dd, J=8.79, 1.95 Hz, 1 H), 8.01 (d, J=8.59 Hz, 1 H), 8.36 (m, 2 H), 10.07 (s, 1 H). MS [M+H) calc. 427.1 found 427.0.

### Example 81

### 1-(4-chlorophenyl)-N-[2-(hydroxymethyl)-1,3-benzothiazol-5-yl]-5-(trifluoromethyl)-1H-pyrazole-4-carboxamide

The title compound was synthesized from 5-amino-2-methylbenzothiazole and 1-(4-chlorophenyl)-5-(trifluoromethyl)-1H-pyrazole-4-carbonyl chloride according to the procedure described in the example 79. ¹H NMR (400 MHz, DMSO-D6) δ ppm 4.86 (d, J=6.05 Hz, 2 H), 6.26 (t, J=5.96 Hz, 1 H), 7.60 (d, J=8.59 Hz, 2 H), 7.69 (m, 3 H), 8.04 (d, J=8.59 Hz, 1 H), 8.37 (m, J=4.69 Hz, 2 H), 10.72 (s, 1 H). MS [M+H) calc. 453.0 found 452.9.

### Example 82

### N-(2,4-dimethyl-1,3-benzothiazol-5-yl)-4-(1-hydroxy-1-methylethyl)benzamide

According to amide bond forming procedure described in Example 1, 5-amino-2-methylbenzothiazole reacted with 4-(methoxycarbonyl)benzoic acid to yield N-(2-Methyl-benzothiazol-5-yl)-terephthalamic acid methyl ester: MS [M+] calc. 326.0, found 326.0. This intermediate was placed into a flask, which was capped with a rubber septum and charged with N₂ gas. THF (10.0 mL) was added, followed by MeMgBr (4.60 mmol, 1.53 mL), and the reaction was stirred for 8 hours at room temperature. A saturated solution of NH₄Cl was added, and the mixture was evaporated to dryness in vacuum. The residue was purified by HPLC eluting with mixtures of MeCN and H₂O containing 1% TFA to yield the title product. 1H NMR (400 MHz, CHLOROFORM-D) δ ppm 1.62 (s, 6 H) 2.96 (s, 3 H) 3.50 (s, 1 H) 7.62 (d, J=8.59 Hz, 2 H) 7.83 (d, J=8.79 Hz, 1 H) 7.90 (d, J=8.59 Hz, 2 H) 8.09 (dd, J=8.79, 1.76 Hz, 2 H) 8.21 (s, 1 H) 8.27 (s, 1 H); MMS [M+] cald. 327.1, found 327.0.

### Example 83

### 4-(Hydroxymethyl)-N-[2-(hydroxymethyl)-1,3-benzothiazol-5-yl]benzamide

According to amide bond forming procedure described in Example 1, allyl (5-amino-1,3-benzothiazol-2-yl)methyl carbonate reacted with p-carboxybenzaldehyde to yield carbonic acid allyl ester 5-(4-formyl-benzoylamino)-benzothiazol-2-ylmethyl ester. This intermediate (97 mg, 0.25 mmol) and B₁₀H₁₄ (30 mg, 0.25 mmol) were stirred in MeOH (10.0 mL) for 48 hours. The reaction mixture was diluted with EtOAc (40.0 mL) and water (30.0 mL) and the organic phase separated. The aqueous phase was extracted with EtOAc (2×10.0 mL) and the combined organic phases were washed with brine solution (30.0 mL). The organic phase was dried with Na₂SO₄, filtered and concentrated in vacuum. The residue was purified by silica gel chromatography (1:1 EtOAc/hexanes) to yield the title product. ¹H NMR (400 MHz, DMSO-D6) δ ppm 4.56 (m, 2 H), 4.83 (m, 2 H), 5.32 (t, J=5.66 Hz, 1 H), 6.21 (t, J=5.96 Hz, 1 H), 7.45 (d, J=8.20 Hz, 2 H), 7.76 (dd, J=8.69, 1.86 Hz, 1 H), 7.94 (d, J=8.20 Hz, 2 H), 7.99 (d, J=8.59 Hz, 1 H), 8.42 (d, J=1.76 Hz, 1 H), 10.35 (s, 1 H). MS [M+H) calc. 315.1 found 315.0.

### Example 84

### 4-tert-butyl-N-(4-cyano-2-methyl-1,3-benzothiazol-5-yl)benzamide

N-(4-Bromo-2-methyl-1,3-benzothiazol-5-yl)-4-tert-butylbenzamide (example 12) (50.0 mg, 0.124 mmol) and CuCN (22 mg, 0.248 mmol) were dissolved in DMF (3.00 mL) and heated to 250° C. in a microwave oven for 20 minutes. The mixture was cooled, and the solvent was evaporated. The residue was purified by flash chromatography on silica gel eluting with mixtures of hexanes and EtOAc (4:1, 2:1, 1:1) to yield the title product. ¹H NMR (400 MHz, chloroform-D) δ ppm 1.36 (s, 9 H) 2.92 (s, 3 H) 7.55 (ddd, J=8.74, 2.25, 2.10 Hz, 2 H) 7.92 (ddd, J=8.64, 2.25, 2.00.Hz, 2 H) 8.03. (d; J=8.98 Hz, 1 H) 8.58 (s, 1 H) 8.69 (m, 1H); MS [M+] calcd. 350.1, found 350.0.

### Example 85

### 4-tert-butyl-N-[2-(hydroxymethyl)-1,3-benzoxazol-5-yl]benzamide

A solution of 2-methyl-5-nitro-1,3-benzoxazole (500 mg, 2.8 mmol) in (dimethoxymethyl)dimethylamine (5.0 ml) was stirred in the microwave at 200° C. for 15 min. (900 sec.). The precipitate was filtered off, washed with methanol and dried yielding (E)-N,N-dimethyl-2-(5-nitro-1,3-benzoxazol-2-yl)ethylenamine, 200 mg (31%), as a yellow powder. ¹H NMR (400 MHz, DMSO-D6) δ ppm 2.72-3.20 (m, 6 H) 5.02 (d, J=13.08 Hz, 1 H) 7.59 (d, J=8.79 Hz, 1 H) 7.74 (d, J=13.08 Hz, 1 H) 8.00 (dd, J=8.79, 2.34 Hz, 1 H) 8.10 (d, J=2.34 Hz, 1 H).

(E)-N,N-Dimethyl-2-(5-nitro-1,3-benzoxazol-2-yl)ethylenamine (200 mg, 0.86 mmol) dissolved in methanol (20 ml), was hydrogenated over 10% palladium on carbon (500 mg) for 1 hour. The catalyst was removed via filtration through Celite and the filtrate was concentrated to yield a crude 2-[(E)-2-(dimethylamino)vinyl]-1,3-benzoxazol-5-amine, 120 mg (69%), which used as such in the next reaction step. ¹H NMR (400 MHz, DMSO-D6) δ ppm 2.88 (s, 6 H) 4.76 (s, 2 H) 4.88 (d, J=13.28 Hz, 1 H) 6.31 (dd, J=8.50, 2.25 Hz, 1 H) 6.54 (d, J=2.25 Hz, 1 H) 7.02 (d, J=8.50 Hz, 1 H) 7.48 (d, J=13.28 Hz, 1 H).

2-[(E)-2-(Dimethylamino)vinyl]-1,3-benzoxazol-5-amine (100 mg, 0.49 mmol) was dissolved in DCM (5.0 ml) containing dimethylaminopyridine (179 mg, 0.74 mmol). 4-tert-Butylbenzoyl chloride (144mg, 1.47 mmol) was added and the mixture was stirred at ambient temperature for 1 h. The mixture was diluted with DCM and extracted with water. The organic phase was dried over anhydrous sodium sulphate, and concentrated under reduced pressure. The residue was purified on a small silica gel column using ethyl acetate as the eluent to yield 4-tert-butyl-N-{2-[(E)-2-(dimethylamino)vinyl]1,3-benzoxazol-5-yl)benzamide, 45 mg (25%). ¹H NMR (400 MHz, methanol-D4) δ ppm 1.37 (s, 9 H) 2.99 (s, 6 H) 5.02 (d, J=13.28 Hz, 1 H) 7.29-7.35 (m, 1 H) 7.35-7.44 (m, 1 H) 7.51-7.57 (m, 2 H) 7.63 (d, J=13.28 Hz, 1 H) 7.78 (d, J=1.95 Hz, 1 H) 7.83-7.93 (m, 2 H).

4-tert-Butyl-N-{2-[(E)-2-(dimethylamino)vinyl]-1,3-benzoxazol-5-yl}benzamide. (45 mg, 0.124 mmol) was dissolved in a mixture of THF and water (1:1, 10 ml) and sodium periodate (158 mg, 0.74 mmol) was added. The mixture was stirred at ambient temperature for 3 h. The solution was extracted with DCM, the organic phase was dried over anhydrous sodium sulphate and concentrated under reduced pressure. The residue was dissolved in methanol (20 ml) and treated with sodium borohydride (200 mg, 5.4 mmol) at ambient temperature for 1 h. The reaction mixture was diluted with water and extracted with dichloromethane. The organic phase was dried over anhydrous sodium sulphate and concentrated under reduced pressure. The residue was purified by a column chromatography on silicagel using 50% ethyl acetate in hexane as an eluent to yield the title product, 18 mg (45%) as colourless oil. ¹H NMR (400 Mz, methanoL-D4) δ ppm 1.35 (s, 9 H) 4.79 (s, 2 H) 7.45-7.64 (m, 3 H) 7.61-7.73 (m, 1 H) 7.80-7.96 (m, 2 H) 8.12 (d, J=1.76 Hz, 1 H). MS [M+] calcd. 325.2, found 325.0.

### Example 86

### 5-(4-tert-butylbenzoylamino)-1,3-benzothiazol-2-ylcarboxylic acid

A solution of 4-tert-butyl-N-(2-formyl-1,3-benzothiazol-5-yl)-benzamide (0.1 mmol) in THF (2 mL) was treated sequentially with a solution of sulfamic acid (0.2 mmol) in water (0.5 mL) and a solution of sodium chlorite (0.15 eq) in water (0.5 mL). The mixture was stirred at ambient temperature for 1 h, then partitioned between ethyl acetate (5 mL ) and water (5 mL). The organic phase was separated, the water phase was extracted 3 times with ethyl acetate. Combined organic phase was dried over anhydrous sodium sulphate and concentrated in vacuo. The crude material was purified by preparative HPLC (X-Terra C8 column, 19×300 mm), using a gradient of A (water 95%, containing NH₄OAc (0.01 M), and 5% acetonitrile) and B (acetonitrile), to give the title compound. MS [M+] calcd. 354.4, found 355.0

### Example 87

### 4-tert-Butyl-N-(2-methoxycarbonyl-1,3-benzothiazol-5-yl)-benzamide

A solution of 5-(4-tert-butylbenzoylamino)-1,3-benzothiazol-2-yl carboxylic acid (0.1 mmol) in methanol (3 mL) was treated with one drop of concentrated hydrochloric acid. The mixture was concentrated to dryness in vacuo. The oily residue was then purified by preparative HPLC (X-Terra C8 column, 19×300 mm), using a gradient of A (water 95%, containing NH₄OAc (0.01 M), and 5% acetonitrile) and B (acetonitrile), to give the title compound as a solid. ¹H NMR (400 MHz, DMSO-d6) δ 1.32 (s, 9 H), 3.65 (s, 1H), 7.25 (d, J=8.6 Hz, 2 H), 7.65-7.79 (m, 2H), 7.85 (d, J=8.6 Hz; 1 H), 7.9.1 .(s, 1 H), 8.29 (s, 1 H). MS [M+] calcd. 368.5, found 369

Pharmacology

1. hVR1 FLIPR (Fluorometric Image Plate Reader) Screening Assay

Transfected CHO cells, stably expessing hVR1 (15,000 cells/well) are seeded in 50 ul media in a black clear bottom 384 plate (Greiner) and grown in a humidified incubator (37° C., 2% CO₂), 24-30 hours prior to experiment.

Subsequently, the media is removed from the cell plate by inversion and 2 μM Fluo-4 is added using a multidrop (Labsystems). Following the 40 minutes dye incubation in the dark at 37° C. and 2% CO₂, the extracellular dye present is washed away using an EMBLA (Scatron), leaving the cells in 40 ul of assay buffer (1×HBSS, 10 mM D-Glucose, 1 mM CaCl₂, 10 mM HEPES, 10×7.5% NaHCO₃ and 2.5 mM Probenecid).

FLIPR Assay—IC₅₀ Determination Protocol

For IC₅₀ determinations the fluorescence is read using FLIPR filter 1 (em 520-545 nM). A cellular baseline recording is taken for 30 seconds, followed by a 20 μl addition of 10, titrated half-log concentrations of the test compound, yielding cellular concentration ranging from 3 μM to 0.1 nM. Data is collected every 2 seconds for a further 5 minutes prior to the addition of a VR1 agonist solution: either 50 nM solution of capsaicin or MES (2-[N-morpholino) ethanesulfonic acid) buffer (pH 5.2), by the FLIPR pipettor. The FLIPR continues to collect data for a further 4 minutes. Compounds having antagonistic properties against the hVR1 will inhibit the increase in intracellular calcium in response to the capsaicin addition. This consequently leading to a reduction in fluorescence signal and providing a reduced fluorescence reading, compared with no compound, buffer controls. Data is exported by the FLIPR program as a sum of fluorescence calculated under the curve upon the addition of capsaicin. Maximum inhibition, Hill slope and IC₅₀ data for each compound are generated.

2. DRGs were dissected out from adult Sprague Dawley rats (100-300 gr), and placed on ice in L15 Leibovitz medium. The ganglia were enzyme treated with Collagenase 80 U/ml+. Dispase 34 U/ml dissolved in DMEM +5% serum, overnight at 37 ° C. The next day, cells were triturated with fire polished pasteur pipettes, and seeded in the center of 58 mm diameter Nunc cell dishes coated with Poly-D Lysine (1 mg/mL). The DRGs were cultured in a defined medium without foetal bovine serum, containing Dulbecco's MEM/NUT MIX F-12 (1:1) without L-glutamine but with pyridoxine, 6 mg/mL D(+)-Glucose, 100 μg/mL apo-transferrin, 1 mg/mL BSA, 20 μg/mL insulin, 2 mM L-glutamine, 50 IU/mL Penicillin, 50 μg/mL Streptomycin and 0.01 μg/mL NGF-7S.

When the cells had grown for 2 days up to 4 weeks, the experiments were done. Cells were chosen based on size and presence of neurites. Small cells with long processes were used for recording (most likely to be C neurons, with native VR1 receptors).

The cells were recorded with conventional whole cell voltage clamp patch clamp, using the following solutions (calcium ion free):

The extracellular solution comprised (in mM): NaCl 137, KCl 5, MgCl₂*H₂O 1.2, HEPES 10, Glucose 10, EGTA 5, Sucrose 50, pH to 7.4 with NaOH.

The intracellular solution comprised K-gluconate 140, NaCl 3, MgCl₂*H₂O 1.2, HEPES 10, EGTA 1, pH to 7.2 with KOH. When the cells were penetrated with suction, a puff of capsaicin (500 nM) was used to determine if the cell expressed VR1 receptor. If not, a new cell was chosen. If yes, then the compounds were added in increasing doses before the capsaicin pulse (500 nM), to determine an IC₅₀ value.

### List of Abbreviations

VR1 vanilloid receptor 1
IBS irritable bowel syndrome
IBD inflammatory bowel disease
GERD gastro-esophageal reflux disease
DRG Dorsal Root Ganglion
BSA Bovine Serum Albumin
HEPES 4-(2-Hydroxyethyl)piperazine-1-ethanesulfonic acid
EGTA Ethylene glycol-bis(2-aminoethylether)-N,N,N′,N′-tetraacetic acid
DMEM Dulbeccos Modified Eagle's Medium

Results

Typical IC₅₀ values as measured in the assays described above are 10 μM or less. In one aspect of the invention the IC₅₀ is below 500 nM. In another aspect of the invention the IC₅₀ is below 100 nM. In a further aspect of the invention the IC₅₀ is below 10 nM.

Results from the hVR1 FLIPR

| | |
|---|---|
| Example No. | IC₅₀ nM (agonist) |

| | | |
|---|---|---|
| 2 | 10 (capsaicin) | 60 (H⁺/MES buffer) |
| 71 | 200 (capsaicin) | |
| 19 | 50 (capsaicin) | 45 (H⁺/MES buffer) |

1-20. (canceled)

21. A compound having the formula I 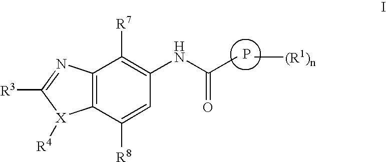
wherein:
ring P is C₆₋₁₀aryl, C₃₋₇cycloalkyl, C₅₋₆-heteroaryl, which ring P may be fused with phenyl, C₅₋₆-heteroaryl, C₃₋₇cycloalkyl or C₃₋₇heterocycloalkyl;
R¹ is NO₂, NH₂, halo, N(C₁₋₆alkyl)₂, C₁₋₆alkyl, C₂₋₆alkenyl, C₂₋₆alkynyl, C₁₋₆haloalkyl, C₁₋₆haloalkylO, phenylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆alkylOC₀₋₆alkyl, C₁₋₆alkylSC₀₋₆alkyl or C₁₋₆alkylNC₀₋₆alkyl;
n is 1, 2, 3, 4 or 5;
X is O, S or N;
R⁴ is H, C₁₋₄alkyl, hydroxyC₁₋₆alkyl or C₁₋₆alkylOC₁₋₆alkyl, or is absent when X is N;
R³ is H, C₁₋₆alkyl, C₁₋₆haloalkyl, R⁵OC₁₋₆alkyl, R⁵O(CO), R⁵CO, NR⁵R⁶CO, NR⁵R⁶C₀₋₆alkyl, C₂₋₆alkenylOC₀₋₆alkyl or hydroxyC₁₋₆alkyl;
R⁵ and R⁶ are at each occurrence independently selected from H, C₁₋₆alkyl, C₆₋₁₀aryl, C₅₋₆heteroaryl, C₁₋₄alkylSO₂ and C₁₋₃alkylCO;
R⁷ and R⁸ are independently selected from H, C₁₋₆alkyl, halo, cyano, C₁₋₆alkylOC₀₋₆alkyl, OH, NO₂ and COR⁹, N(R⁹)₂; and
R⁹ is H or C₁₋₆alkyl;
wherein any alkyl, alkylOalkyl, haloalkyl, haloalkylO, phenyl, heteroaryl, cycloalkyl or heterocycloalkyl group may be substituted with one or more A, where A at each occurrence is independently selected from OH, NO₂, R⁹CO, R⁹O(CO), N(R⁹)₂, R⁹S, R⁹SO₂, halo or C₁₋₆alkylOC₀₋₆alkyl,
or a salt, solvate or solvated salt thereof.

22. A compound according to claim 21, wherein:
X is N;
R³ is H, C₁₋₆alkyl, C₁₋₆iodoalkyl, C₁₋₆bromoalkyl, C₁₋₆chloroalkyl, C₁₋₆alkylOC₀₋₆alkyl, R⁵OC₁₋₆alkyl, R⁵CO, R⁵CO₂, NR⁵R⁶CO, NR⁵R⁶C₀₋₆alkyl or C₂₋₆alkenylOC₀₋₆alkyl; and
R⁴ is H, C₁₋₄alkyl, hydroxyC₁₋₆alkyl or C₁₋₆alkylOC₁₋₆alkyl.

23. A compound according to claim 21, wherein:
X is N;
R³ is C₁₋₆fluoroalkyl or hydroxyC₁₋₂alkyl, and
R⁴ is H.

24. A compound according to claim 21, wherein:
X is O or S;
R³ is H, C₁₋₆alkyl, C₁₋₆haloalkyl, R⁵OC₁₋₆alkyl, R⁵O(CO), R⁵CO, NR⁵R⁶CO, NR⁵R⁶C₀₋₆alkyl, C₂₋₆alkenylOC₀₋₆alkyl or hydroxyC₁₋₆alkyl, and R⁴ is absent.

25. A compound according to claim 21, wherein:
ring P is C₆₋₁₀aryl, C₅₋₆heteroaryl, which ring P may be fused with C₃₋₇heterocycloalkyl;
R¹ is NO₂, NH₂, halo, N(C₁₋₆alkyl)₂, C₁₋₆alkyl, C₂₋₆alkenyl, C₁₋₆haloalkyl, C₁₋₆haloalkylO, phenylC₀₋₆alkyl, C₃₋₇heterocycloalkylC₀₋₆alkyl, C₁₋₆alkylOC₀₋₆alkyl or C₁₋₆alkylSC₀₋₆alkyl;
n is 1, 2 or 3;
X is O, N or S;
R⁴ is C₁₋₄alkyl or hydroxyC₁₋₆alkyl, or is absent when X is N;
R³ is C₁₋₆alkyl, NR⁵R⁶CO, NR⁵R⁶C₀₋₆alkyl, C₂₋₆alkenylOC₀₋₆alkyl or hydroxyC₁₋₆alkyl;
R⁵ and R⁶ are independently selected from H, C₆₋₁₀aryl, C₅₋₆heteroaryl, C₁₋₄alkylSO₂ and C₁₋₃alkylCO;
R⁷ and R⁸ are independently selected from H, halo and cyano and wherein:
any alkyl, phenyl, heteroaryl group may be substituted with one or more A where A at each occurrence is independently selected from OH, NO₂, halo or C₁₋₆alkylOC₀₋₆alkyl.

26. A compound according to claim 25, wherein:
X is N;
R³ is H or C₁₋₆alkyl; and
R⁴ is H.

27. A compound according to claim 25, wherein:
X is N;
R³ is C₁₋₆fluoroalkyl and
R⁴ is H.

28. A compound according to claim 21, wherein X is S and R³is methyl.

29. A compound according to claim 21, wherein X is O and R³ is C₁₋₆alkyl or hydroxyC₁₋₆alkyl.

30. A compound according to claim 21, wherein X is N, R³ is C₁₋₆alkyl and R⁴ is C₁₋₆alkyl or hydroxyC₁₋₆alkyl.

31. A compound according to claim 21, wherein:
ring P is phenyl, and R¹ is NO₂, NH₂, halo, N(C₁₋₆alkyl)₂, C₁₋₆alkyl, C₂₋₆alkenyl, C₂₋₆alkynyl, C₁₋₆haloalkyl, C₁₋₆haloalkylO, phenylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆alkylOC₀₋₆alkyl, C₁₋₆alkylSC₀₋₆alkyl or C₁₋₆alkylNC₀₋₆alkyl optionally substituted with one or more A.

32. A compound according to claim 21, wherein
ring P is pyrazolyl, pyridine, benzdioxolane, furan, thiophene or naphthalene.

33. A compound according to claim 21, selected from the group consisting of:
4-tert-Butoxy-N-[2-(hydroxymethyl)-1,3-benzothiazol-5-yl]benzamide;
4-Bromo-N-[2-(hydroxymethyl)-1,3-benzothiazol-5-yl]benzamide;
N-[2-(Hydroxymethyl)-1,3-benzothiazol-5-yl]-4-iodobenzamide;
N-[2-(Hydroxymethyl)-1,3-benzothiazol-5-yl]-4-morpholin-4-ylbenzamide;
N-{2-[(Allyloxy)methyl]-1,3-benzothiazol-5-yl}-4-morpholin-4-ylbenzamide;
N-[2-(Hydroxymethyl)-1,3-benzothiazol-5-yl]-1-phenyl-5-propyl-1H-pyrazole-4-carboxamide;
1-tert-Butyl-N-[2-(hydroxymethyl)-1,3-benzothiazol-5-yl]-3-methyl-1H-pyrazole-5-carboxamide;
4-(Ethoxymethyl)-N-[2-(hydroxymethyl)-1,3-benzothiazol-5-yl]benzamide;
N-[2-(Hydroxymethyl)-1,3-benzothiazol-5-yl]-1-phenyl-1H-pyrazole-5-carboxamide;
4-Bromo-N-[2-(hydroxymethyl)-1,3-benzothiazol-5-yl]-2-methylbenzamide;
4-tert-Butoxy-N-(2-methyl-1,3-benzoxazol-5-yl) benzamide;
N-(4-Bromo-2-methyl-1,3-benzothiazol-5-yl)-4-tert-butylbenzamide;
4-tert-Butyl-N-(4,7-dibromo-2-methyl-1,3-benzothiazol-5-yl)benzamide;
N-[2-(Hydroxymethyl)-1,3-benzothiazol-5-yl]-1-phenyl-5-(trifluoromethyl)-1H-pyrazole-4-carboxamide;
4-Iodo-N-(2-methyl-5-benzothiazolyl)benzamide;
4-(tert-Butoxymethyl)-N-[2-(hydroxymethyl)-1,3-benzothiazol-5-yl]benzamide;
N-( 1,2-Dimethyl-1H-benzimidazol-5-yl)-4-iodobenzamide;
N-[2-(Hydroxymethyl)-1,3-benzothiazol-5-yl]-4-[2,2,2-trifluoro-1-hydroxy-1-(trifluoromethyl)ethyl]benzamide;
N-[2-(Hydroxymethyl)-1,3-benzothiazol-5-yl]-4-isopropoxybenzamide;
4-Bromo-2-chloro-N-[2-(hydroxymethyl)-1,3-benzothiazol-5-yl]benzamide;
4-Bromo-2-fluoro-N-[2-(hydroxymethyl)-1,3-benzothiazol-5-yl]benzamide;
N-[2-(Hydroxymethyl)-1,3-benzothiazol-5-yl]-4-(morpholin-4-ylmethyl)benzamide;
3-Fluoro-N-[2-(hydroxymethyl)-1,3-benzothiazol-5-yl]-4-(trifluoromethyl)benzamide;
4-tert-Butoxy-N-[4-chloro-2-(hydroxymethyl)-1,3-benzothiazol-5-yl]benzamide;
4-(tert-Butoxymethyl)-N-[4-chloro-2-(hydroxymethyl)-1,3-benzothiazol-5-yl]benzamide;
3-Fluoro-N-(2-methyl-1,3-benzothiazol-5-yl)-4-trifluoromethyl-benzamide;
2-tert-Butyl-5-methyl-2H-pyrazole-3-carboxylic acid (2-methyl-1,3-benzothiazol-5-yl)-amide;
2-Fluoro-N-(2-methyl-1,3-benzothiazol-5-yl)-4-trifluoromethyl-benzamide;
2-Fluoro-N-(2-methyl-1,3-benzothiazol-5-yl)-3-trifluoromethyl-benzamide;
4-Fluoro-N-(2-methyl-1,3-benzothiazol-5-yl)-3-trifluoromethyl-benzamide;
3,4-Dimethyl-N-(2-methyl-benzothiazol-5-yl)-benzamide;
2,2-Difluoro-benzo[1,3]dioxole-5-carboxylic acid (2-methyl-1,3-benzothiazol-5-yl)-amide;
N-(2-Methyl-1,3-benzothiazol-5-yl)-6-trifluoromethyl-nicotinamide;
N-(2-Methyl-1,3-benzothiazol-5-yl)-4-propyl-benzamide;
3-Iodo-N-(2-methyl-1,3-benzothiazol-5-yl)-benzamide;
2,5-Dimethyl-furan-3-carboxylic acid (2-methyl-1,3-benzothiazol-5-yl)-amide;
5-tert-Butyl-2-methyl-furan-3-carboxylic acid (2-methyl-1,3-benzothiazol-5-yl)-amide;
4-Bromo-3-methyl-N-(2-methyl-1,3-benzothiazol-5-yl)-benzamide;
3,4-Difluoro-N-(2-methyl-1,3-benzothiazol-5-yl)-benzamide;
3-Chloro-2-fluoro-N-(2-methyl-1,3-benzothiazol-5-yl)-benzamide;
Pyridine-2-carboxylic acid (2-methyl-1,3-benzothiazol-5-yl)-amide;
2-Benzyl-5-tert-butyl-2H-pyrazole-3-carboxylic acid (2-methyl-1,3-benzothiazol-5-yl)-amide;
3-Fluoro-4-trifluoromethyl-N-(2-trifluoromethyl-1H-benzimidazol-5-yl)-benzamide;
2-Fluoro-5-trifluoromethyl-N-(2-trifluoromethyl-1H-benzimidazol-5-yl)-benzamide;
4-Chloro-N-(2-methyl-benzothiazol-5-yl)-benzamide;
1-Phenyl-5-trifluoromethyl-1H-pyrazole-3-carboxylic acid (2-methyl-1,3-benzothiazol-5-yl)-amide;
1-Phenyl-5-propyl-1H-pyrazole-4-carboxylic acid (2-methyl-1,3-benzothiazol-5-yl)-amide;
2,3-Difluoro-N-(2-methyl-1,3-benzothiazol-5-yl)-4-trifluoromethyl-benzamide;
3-Fluoro-4-methyl-N-(2-methyl-1,3-benzothiazol-5-yl)-benzamide;
4-tert-Butyl-N-(2-methyl-1,3-benzothiazol-5-yl)-benzamide;
4-Ethyl-N-(2-methyl-1,3-benzothiazol-5-yl)-benzamide;
4-tert-Butyl-N-(2-methyl-1,3-benzooxazol-5-yl)-benzamide;
Biphenyl-4-carboxylic acid (2-methyl-1,3-benzothiazol-5-yl)-amide;
3-Bromo-thiophene-2-carboxylic acid (2-methyl-1,3-benzothiazol-5-yl)-amide;
4-Bromo-2-methyl-N-(2-methyl-1,3-benzothiazol-5-yl)-benzamide;
4-tert-Butoxy-N-(2-methyl-1,3-benzothiazol-5-yl)-benzamide;
2-Chloro-3,4-dimethoxy-N-(2-methyl-1,3-benzothiazol-5-yl)-benzamide;
4-Iodo-N-(2-methyl-1,3-benzothiazol-5-yl)-benzamide;
4-Amino-N-(2-methyl-1,3-benzothiazol-5-yl)-3-nitro-benzamide;
N-(2-Methyl-1,3-benzothiazol-5-yl)-4-vinyl-benzamide;
4-Ethoxy-N-(2-methyl-1,3-benzothiazol-5-yl)-benzamide;
4-Ethylsulfanyl-N-(2-methyl-1,3-benzothiazol-5-yl)-benzamide;
4-Dimethylamino-naphthalene-1-carboxylic acid (2-methyl-1,3-benzothiazol-5-yl)-amide;
2-Fluoro-6-iodo-N-(2-methyl-1,3-benzothiazol-5-yl)-benzamide;
4-Ethoxymethyl-N-(2-methyl-1,3-benzothiazol-5-yl)-benzamide;
N-(2-Methyl-1,3-benzothiazol-5-yl)-4-trifluoromethoxy-benzamide;
4-Chloro-3-fluoro-N-(2-methyl-1,3-benzothiazol-5-yl)-benzamide;
4-tert-Butyl-N-(2-formyl-1,3-benzothiazol-5-yl)-benzamide;
4-tert-Butyl-N-(2-hydroxymethyl-1,3-benzothiazol-5-yl)-benzamide;
4-tert-Butyl-N-(2-{[(2-methoxypyridin-3-yl)amino]methyl}-1,3-benzothiazol-5-yl)benzamide;
4-tert-Butyl-N-[2-( 1-hydroxyethyl)-1,3-benzothiazol-5-yl]benzamide;
4-tert-Butyl-N-{2-[(1H-pyrazol-3-ylamino)methyl]-1,3-benzothiazol-5-yl}benzamide;
4-(1,1-Dimethylethyl)-N-[2-[[(4-nitrophenyl)amino]methyl]-5-benzothiazolyl]-benzamide;
N-[2-(Aminomethyl)-1,3-benzothiazol-5-yl]-4-tert-butylbenzamide;
4-tert-Butyl-N-(2-{[(methylsulfonyl)amino]methyl}-1,3-benzothiazol-5-yl)benzamide;
N-{2-[(Acetylamino)methyl]-1,3-benzothiazol-5-yl}-4-tert-butylbenzamide;
5-[(4-tert-Butylbenzoyl)amino]-1,3-benzothiazole-2-carboxamide;
N-1,3-Benzothiazol-5-yl-4-tert-butylbenzamide;
4-Chloro-N-[2-(hydroxymethyl)-1,3-benzothiazol-5-yl]benzamide;
1-(4-chlorophenyl)-N-[2-(hydroxymethyl)-1,3-benzothiazol-5-yl]-5-propyl-1H-pyrazole-4-carboxamide;
1-(4-Chlorophenyl)-N-[2-(hydroxymethyl)-1,3-benzothiazol-5-yl]-5-(trifluoromethyl)-1H-pyrazole-4-carboxamide;
N-(2,4-Dimethyl-1,3-benzothiazol-5-yl)-4-(1-hydroxy-1-methylethyl)benzamide;
4-(Hydroxymethyl)-N-[2-(hydroxymethyl)-1,3-benzothiazol-5-yl]benzamide;
4-tert-Butyl-N-(4-cyano-2-methyl-1,3-benzothiazol-5-yl)benzamide;
4-tert-Butyl-N-[2-(hydroxymethyl)-1,3-benzoxazol-5-yl]benzamide;
5-(4-tert-Butylbenzoylamino)-1,3-benzothiazol-2-ylcarboxylic acid, and
4-tert-Butyl-N-(2-methoxycarbonyl-1,3-benzothiazol-5-yl)-benzamide;
or a salt, solvate or solvated salt thereof.

34. A pharmaceutical composition comprising as an active ingredient a therapeutically effective amount of compound having the formula I 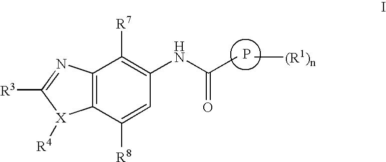
wherein:
ring P is C₆₋₁₀aryl, C₃₋₇cycloalkyl, C₅₋₆heteroaryl, which ring P may be fused with phenyl, C₅₋₆heteroaryl, C₃₋₇cycloalkyl or C₃₋₇heterocycloalkyl;
R¹ is NO₂, NH₂, halo, N(C₁₋₆alkyl)₂, C₁₋₆alkyl, C₂₋₆alkenyl, C₂₋₆alkynyl, C₁₋₆haloalkyl, C₁₋₆haloalkylO, phenylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆alkylOC₀₋₆alkyl, C₁₋₆alkylSC₀₋₆alkyl or C₁₋₆alkylNC₀₋₆alkyl
n is 1,2, 3, 4 or 5;
X is O, S or N;
R⁴ is H, C₁₋₄alkyl, hydroxyC₁₋₆alkyl or C₁₋₆alkylOC₁₋₆alkyl, or is absent when X is N;
R³ is H, C₁₋₆alkyl, C₁₋₆haloalkyl, R⁵OC₁₋₆alkyl, R⁵O(CO), R⁵CO, NR⁵R⁶CO, NR⁵R⁶C₀₋₆alkyl, C₂₋₆alkenylOC₀₋₆alkyl or hydroxyC₁₋₆alkyl;
R⁵ and R⁶ are at each occurrence independently selected from H, C₁₋₆alkyl, C₆₋₁₀aryl, C₅₋₆heteroaryl, C₁₋₄alkylSO₂ and C₁₋₃alkylCO;
R⁷ and R⁸ are independently selected from H, C₁₋₆alkyl, halo, cyano, C₁₋₆alkylOC₀₋₆alkyl, OH, NO₂ and COR⁹, N(R⁹)₂; and
R⁹ is H or C₁₋₆alkyl;
wherein any alkyl, alkylOalkyl, haloalkyl, haloalkylO, phenyl, heteroaryl, cycloalkyl or heterocycloalkyl group may be substituted with one or more A, where A at each occurrence is independently selected from OH, NO₂, R⁹CO, R⁹O(CO), N(R⁹)₂, R⁹S, R⁹SO₂, halo or C₁₋₆alkylOC₀₋₆alkyl,
or a salt, solvate or solvated salt thereof,
in association with one or more pharmaceutically acceptable diluents, excipients or inert carriers.

35. A method of treating VR1 mediated disorders, acute and chronic pain disorders, acute and chronic neuropathic pain or acute and chronic inflammatory pain comprising administering a therapeutically effective amount of a pharmaceutical composition according to claim 34.

36. A method of treatment of VR1 mediated disorders, acute and chronic pain disorders, acute and chronic neuropathic pain, acute or chronic inflammatory pain, or respiratory diseases, comprising administering to a mammal a therapeutically effective amount of compound having the formula I 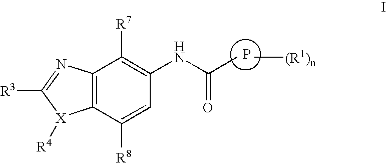
wherein:
ring P is C₆₋₁₀aryl, C₃₋₇cycloalkyl, C₅₋₆heteroaryl, which ring P may be fused with phenyl, C₅₋₆heteroaryl, C₃₋₇cycloalkyl or C₃₋₇heterocycloalkyl;
R¹ is NO₂, NH₂, halo, N(C₁₋₆alkyl)₂, C₁₋₆alkyl, C₂₋₆alkenyl, C₂₋₆alkynyl, C₁₋₆haloalkyl, C₁₋₆haloalkylO, phenylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆alkylOC₀₋₆alkyl, C₁₋₆alkylSC₀₋₆alkyl or C₁₋₆alkylNC₀₋₆alkyl;
n is 1,2,3,4 or 5;
X is O, S or N;
R⁴ is H, C₁₋₄alkyl, hydroxyC₁₋₆alkyl or C₁₋₆alkylOC₁₋₆alkyl, or is absent when X is N;
R³ is H, C₁₋₆alkyl, C₁₋₆haloalkyl, R⁵OC₁₋₆alkyl, R⁵O(CO), R⁵CO, NR⁵R⁶CO, NR⁵R⁶C₀₋₆alkyl, C₂₋₆alkenylOC₀₋₆alkyl or hydroxyC₁₋₆alkyl;
R⁵ and R⁶ are at each occurrence independently selected from H, C₁₋₆alkyl, C_{6 10}aryl, C₅₋₆heteroaryl, C₁₋₄alkylSO₂ and C₁₋₃alkylCO;
R⁷ and R⁸ are independently selected from H, C₁₋₆alkyl, halo, cyano, C₁₋₆alkylOC₀₋₆alkyl, OH, NO₂ and COR⁹, N(R⁹)₂; and
R⁹ is H or C₁₋₆alkyl;
wherein any alkyl, alkylOalkyl, haloalkyl, haloalkylO, phenyl, heteroaryl, cycloalkyl or heterocycloalkyl group may be substituted with one or more A, where A at each occurrence is independently selected from OH, NO₂, R⁹CO, R⁹O(CO), N(R⁹)₂, R⁹S, R⁹SO₂, halo or C₁₋₆alkylOC₀₋₆alkyl,
or a salt, solvate or solvated salt thereof.

37. The method according to claim 36 wherein said mammal is a human.

38. A compound selected from the group consisting of
Allyl (5-amino-1,3-benzothiazol-2-yl)methyl carbonate;
4-tert-Butyl-N-(2-formyl-1,3-benzothiazol-5-yl)-benzamide;
4-Bromo-2-methyl-benzothiazol-5-ylamine, and
4-Chloro-2-methyl-benzothiazole-5-ylamine.

39. A pharmaceutical composition comprising as an active ingredient a therapeutically effective amount of the group consisting of:
4-tert-Butoxy-N-[2-(hydroxymethyl)-1,3-benzothiazol-5-yl]benzamide;
4-Bromo-N-[2-(hydroxymethyl)-1,3-benzothiazol-5-yl]benzamide;
N-[2-(Hydroxymethyl)-1,3-benzothiazol-5-yl]-4-iodobenzamide;
N-[2-(Hydroxymethyl)-1,3-benzothiazol-5-yl]-4-morpholin-4-ylbenzamide;
N-{2-[(Allyloxy)methyl]-1,3-benzothiazol-5-yl}-4-morpholin-4-ylbenzamide; N-[2-(Hydroxymethyl)-1,3-benzothiazol-5-yl]-1-phenyl-5-propyl-1H-pyrazole-4-carboxamide;
1-tert-Butyl-N-[2-(hydroxymethyl)-1,3-benzothiazol-5-yl]-3-methyl-1H-pyrazole-5-carboxamide;
4-(Ethoxymethyl)-N-[2-(hydroxymethyl)-1,3-benzothiazol-5-yl]benzamide;
N-[2-(Hydroxymethyl)-1,3-benzothiazol-5-yl]-1-phenyl-1H-pyrazole-5-carboxamide;
4-Bromo-N-[2-(hydroxymethyl)-1,3-benzothiazol-5-yl]-2-methylbenzamide;
4-tert-Butoxy-N-(2-methyl-1,3-benzoxazol-5-yl)benzamide;
N-(4-Bromo-2-methyl-1,3-benzothiazol-5-yl)-4-tert-butylbenzamide;
4-tert-Butyl-N-(4,7-dibromo-2-methyl-1,3-benzothiazol-5-yl)benzamide;
N-[2-(Hydroxymethyl)-1,3-benzothiazol-5-yl]-1-phenyl-5-(trifluoromethyl)-1H-pyrazole-4-carboxamide;
4-Iodo-N-(2-methyl-5-benzothiazolyl)benzamide;
4-(tert-Butoxymethyl)-N-[2-(hydroxymethyl)-1,3-benzothiazol-5-yl]benzamide;
N-( 1,2-Dimethyl-1H-benzimidazol-5-yl)-4-iodobenzamide;
N-[2-(Hydroxymethyl)-1,3-benzothiazol-5-yl]-4-[2,2,2-trifluoro-1-hydroxy-1-(trifluoromethyl)ethyl]benzamide;
N-[2-(Hydroxymethyl)-1,3-benzothiazol-5-yl]-4-isopropoxybenzamide;
4-Bromo-2-chloro-N-[2-(hydroxymethyl)-1,3-benzothiazol-5-yl]benzamide;
4-Bromo-2-fluoro-N-[2-(hydroxymethyl)-1,3-benzothiazol-5-yl]benzamide;
N-[2-(Hydroxymethyl)-1,3-benzothiazol-5-yl]-4-(morpholin-4-ylmethyl)benzamide;
3-Fluoro-N-[2-(hydroxymethyl)-1,3-benzothiazol-5-yl]-4-(trifluoromethyl)benzamide;
4-tert-Butoxy-N-[4-chloro-2-(hydroxymethyl)-1,3-benzothiazol-5-yl]benzamide;
4-(tert-Butoxymethyl)-N-[4-chloro-2-(hydroxymethyl)-1,3-benzothiazol-5-yl]benzamide;
3-Fluoro-N-(2-methyl-1,3-benzothiazol-5-yl)-4-trifluoromethyl-benzamide;
2-tert-Butyl-5-methyl-2H-pyrazole-3-carboxylic acid (2-methyl-1,3-benzothiazol-5-yl)-amide;
2-Fluoro-N-(2-methyl-1,3-benzothiazol-5-yl)-4-trifluoromethyl-benzamide;
2-Fluoro-N-(2-methyl-1,3-benzothiazol-5-yl)-3-trifluoromethyl-benzamide;
4-Fluoro-N-(2-methyl-1,3-benzothiazol-5-yl)-3-trifluoromethyl-benzamide;
3,4-Dimethyl-N-(2-methyl-benzothiazol-5-yl)-benzamide;
2,2-Difluoro-benzo[1,3]dioxole-5-carboxylic acid (2-methyl-1,3-benzothiazol-5-yl)-amide;
N-(2-Methyl-1,3-benzothiazol-5-yl)-6-trifluoromethyl-nicotinamide;
N-(2-Methyl-1,3-benzothiazol-5-yl)-4-propyl-benzamide;
3-Iodo-N-(2-methyl-1,3-benzothiazol-5-yl)-benzamide;
2,5-Dimethyl-furan-3-carboxylic acid (2-methyl-1,3-benzothiazol-5-yl)-amide;
5-tert-Butyl-2-methyl-furan-3-carboxylic acid (2-methyl-1,3-benzothiazol-5-yl)-amide;
4-Bromo-3-methyl-N-(2-methyl-1,3-benzothiazol-5-yl)-benzamide;
3,4-Difluoro-N-(2-methyl-1,3-benzothiazol-5-yl)-benzamide;
3-Chloro-2-fluoro-N-(2-methyl-1,3-benzothiazol-5-yl)-benzamide;
Pyridine-2-carboxylic acid (2-methyl-1,3-benzothiazol-5-yl)-amide;
2-Benzyl-5-tert-butyl-2H-pyrazole-3-carboxylic acid (2-methyl-1,3-benzothiazol-5-yl)-amide;
3-Fluoro-4-trifluoromethyl-N-(2-trifluoromethyl-1H-benzimidazol-5-yl)-benzamide;
2-Fluoro-5-trifluoromethyl-N-(2-trifluoromethyl-1H-benzimidazol-5-yl)-benzamide;
4-Chloro-N-(2-methyl-benzothiazol-5-yl)-benzamide;
1-Phenyl-5-trifluoromethyl-1H-pyrazole-3-carboxylic acid (2-methyl-1,3-benzothiazol-5-yl)-amide;
1-Phenyl-5-propyl-1H-pyrazole-4-carboxylic acid (2-methyl-1,3-benzothiazol-5-yl)-amide;
2,3-Difluoro-N-(2-methyl-1,3-benzothiazol-5-yl)-4-trifluoromethyl-benzamide;
3-Fluoro-4-methyl-N-(2-methyl-1,3-benzothiazol-5-yl)-benzamide;
4-tert-Butyl-N-(2-methyl-1,3-benzothiazol-5-yl)-benzamide;
4-Ethyl-N-(2-methyl-1,3-benzothiazol-5-yl)-benzamide;
4-tert-Butyl-N-(2-methyl-1,3-benzooxazol-5-yl)-benzamide;
Biphenyl-4-carboxylic acid (2-methyl-1,3-benzothiazol-5-yl)-amide;
3-Bromo-thiophene-2-carboxylic acid (2-methyl-1,3-benzothiazol-5-yl)-amide;
4-Bromo-2-methyl-N-(2-methyl-1,3-benzothiazol-5-yl)-benzamide;
4-tert-Butoxy-N-(2-methyl-1,3-benzothiazol-5-yl)-benzamide;
2-Chloro-3,4-dimethoxy-N-(2-methyl-1,3-benzothiazol-5-yl)-benzamide;
4-Iodo-N-(2-methyl-1,3-benzothiazol-5-yl)-benzamide;
4-Amino-N-(2-methyl-1,3-benzothiazol-5-yl)-3-nitro-benzamide;
N-(2-Methyl-1,3-benzothiazol-5-yl)-4-vinyl-benzamide;
4-Ethoxy-N-(2-methyl-1,3-benzothiazol-5-yl)-benzamide;
4-Ethylsulfanyl-N-(2-methyl-1,3-benzothiazol-5-yl)-benzamide;
4-Dimethylamino-naphthalene-1-carboxylic acid (2-methyl-1,3-benzothiazol-5-yl)-amide;
2-Fluoro-6-iodo-N-(2-methyl-1,3-benzothiazol-5-yl)-benzamide;
4-Ethoxymethyl-N-(2-methyl-1,3-benzothiazol-5-yl)-benzamide;
N-(2-Methyl-1,3-benzothiazol-5-yl)-4-trifluoromethoxy-benzamide;
4-Chloro-3-fluoro-N-(2-methyl-1,3-benzothiazol-5-yl)-benzamide;
4-tert-Butyl-N-(2-formyl-1,3-benzothiazol-5-yl)-benzamide;
4-tert-Butyl-N-(2-hydroxymethyl-1,3-benzothiazol-5-yl)-benzamide;
4-tert-Butyl-N-(2-{[(2-methoxypyridin-3-yl)amino]methyl}-1,3-benzothiazol-5-yl)benzamide;
4-tert-Butyl-N-[2-( 1-hydroxyethyl)-1,3-benzothiazol-5-yl]benzamide;
4-tert-Butyl-N-{2-[(1H-pyrazol-3-ylamino)methyl]-1,3-benzothiazol-5-yl}benzamide;
4-(1,1-Dimethylethyl)-N-[2-[[(4-nitrophenyl)amino]methyl]-5-benzothiazolyl]-benzamide;
N-[2-(Aminomethyl)-1,3-benzothiazol-5-yl]-4-tert-butylbenzamide;
4-tert-Butyl-N-(2-{[(methylsulfonyl)amino]methyl}-1,3-benzothiazol-5-yl)benzamide;
N-{2-[(Acetylamino)methyl]-1,3-benzothiazol-5-yl}-4-tert-butylbenzamide;
5-[(4-tert-Butylbenzoyl)amino]-1,3-benzothiazole-2-carboxamide;
N-1,3-Benzothiazol-5-yl-4-tert-butylbenzamide;
4-Chloro-N-[2-(hydroxymethyl)-1,3-benzothiazol-5-yl]benzamide;
1-(4-chlorophenyl)-N-[2-(hydroxymethyl)-1,3-benzothiazol-5-yl]-5-propyl-1H-pyrazole-4carboxamide;
1-(4-Chlorophenyl)-N-[2-(hydroxymethyl)-1,3-benzothiazol-5-yl]-5-(trifluoromethyl)-1H-pyrazole-4-carboxamide;
N-(2,4-Dimethyl-1,3-benzothiazol-5-yl)-4-( 1-hydroxy-1-methylethyl)benzamide;
4-(Hydroxymethyl)-N-[2-(hydroxymethyl)-1,3-benzothiazol-5-yl]benzamide;
4-tert-Butyl-N-(4-cyano-2-methyl-1,3-benzothiazol-5-yl)benzamide;
4-tert-Butyl-N-[2-(hydroxymethyl)-1,3-benzoxazol-5-yl]benzamide;
5-(4-tert-Butylbenzoylamino)-1,3-benzothiazol-2-ylcarboxylic acid, and
4-tert-Butyl-N-(2-methoxycarbonyl-1,3-benzothiazol-5-yl)-benzamide;
or a salt, solvate or solvated salt thereof,
in association with one or more pharmaceutically acceptable diluents, excipients or inert carriers.

40. A method of treating VR1 mediated disorders, acute and chronic pain disorders, acute and chronic neuropathic pain or acute and chronic inflammatory pain comprising administering a therapeutically effective amount of a pharmaceutical composition according to claim 39.
